SEQUENCE LISTING (30 sequences)

SEQ ID NO: 1 - DNA, 1416 bp, reovirus
gctattggtcggatggatcctcgcctacgtgaagaagtagtacggctgataatcgcattaacgagtgataatggagcatcactgtcaaaagggcttgaatcaagggtctcggcgctcgagaagacgtctcaaatacactctgatactatcctccggatcacccagggactcgatgatgcaaacaaacgaatcatcgctcttgagcaaagtcgggatgacttggttgcatcagtcagtgatgctcaacttgcaatctccagattggaaagctctatcggagccctccaaacagttgtcaatggacttgattcgagtgttacccagttgggtgctcgagtgggacaacttgagacaggacttgcagagctacgcgttgatcacgacaatctcgttgcgagagtggatactgcagaacgtaacattggatcattgaccactgagctatcaactctgacgttacgagtaacatccatacaagcggatttcgaatctaggatatccacgttagagcgcacggcggtcactagcgcgggagctcccctctcaatccgtaataaccgtatgaccatgggattaaatgatggactcacgttgtcagggaataatctcgccatccgattgccaggaaatacgggtctgaatattcaaaatggtggacttcagtttcgatttaatactgatcaattccagatagttaataataacttgactctcaagacgactgtgtttgattctatcaactcaaggataggcgcaactgagcaaagttacgtggcgtcggcagtgactcccttgagattaaacagtagcacgaaggtgctggatatgctaatagacagttcaacacttgaaattaattctagtggacagctaactgttagatcgacatccccgaatttgaggtatccgatagctgatgttagcggcggtatcggaatgagtccaaattataggtttaggcagagcatgtggataggaattgtctcctattctggtagtgggctgaattggagggtacaggtgaactccgacatttttattgtagatgattacatacatatatgtcttccagcttttgacggtttctctatagctgacggtggagatctatcgttgaactttgttaccggattgttaccaccgttacttacaggagacactgagcccgcttttcataatgacgtggtcacatatggagcacagactgtagctatagggttgtcgtcgggtggtgcgcctcagtatatgagtaagaatctgtgggtggagcagtggcaggatggagtacttcggttacgtgttgaggggggtggctcaattacgcactcaaacagtaagtggcctgccatgaccgtttcgtacccgcgtagtttcacgtgaggatcagaccaccccgcggcactggggcatttcatc

SEQ ID NO: 2 - DNA, 1331 bp, reovirus
gctattcgctggtcagttatggctcgcgctgcgttcctattcaagactgttgggtttggtggtctgcaaaatgtgccaattaacgacgaactatcttcacatctactccgagctggtaattcaccatggcagttaacacagtttttagactggataagccttgggaggggtttagctacatcggctctcgttccgacggctgggtcaagatactatcaaatgagttgccttctaagtggcactctccagattccgttccgtcctaaccaccgatggggagacattaggttcttacgcttagtgtggtcagctcctactctcgatggattagtcgtagctccaccacaagttttggctcagcccgctttgcaagcacaggcagatcgagtgtacgactgcgatgattatccatttctagcgcgtgatccaagattcaaacatcgggtgtatcagcaattgagtgctgtaactctacttaacttgacaggttttggcccgatttcctacgttcgagtggatgaagatatgtggagtggagatgtgaaccagcttctcatgaactatttcgggcacacgtttgcagagattgcatacacattgtgtcaagcctcggctaataggccttgggaatatgacggtacatatgctaggatgactcagattgtgttatccttgttctggctatcgtatgtcggtgtaattcatcagcagaatacgtatcggacattctattttcagtgtaatcggcgaggtgacgccgctgaggtgtggattctttcttgttcgttgaaccattccgcacaaattagaccgggtaatcgtagcttattcgttatgccaactagcccagattggaacatggacgtcaatttgatcctgagttcaacgttgacggggtgtttgtgttcgggttcacagctgccactgattgacaataattcagtacctgcagtgtcgcgtaacatccatggctggactggtagagctggtaaccaattgcatgggttccaggtgagacgaatggtgactgaattttgtgacaggttgagacgcgatggtgtcatgacccaagctcagcagaatcaagttgaagcgttggcagatcagactcaacagtttaagagggacaagctcgaaacgtgggcgagagaagacgatcaatataatcaggctcatcccaactccacaatgttccgtacgaaaccatttacgaatgcgcaatggggacgaggtaatacgggggcgactagtgccgcgattgcagcccttatctgatcgtcttggagtgagggggtccccccacacacctcacgactgaccacacattcatc

SEQ ID NO: 3 - DNA, 1198 bp, reovirus
gctaaagtcacgcctgtcgtcgtcactatggcttcctcactcagagctgcgatctccaagatcaagagggatgacgtcggtcagcaagtttgtcctaattatgtcatgctgcggtcctctgtcacaacaaaggtggtacgaaatgtggttgagtatcaaattcgtacgggcggattcttttcgtgcttagctatgctaaggccactccagtacgctaagcgtgagcgtttgcttggtcagaggaatctggaacgtatatcgactagggatatccttcagactcgtgatttacactcactatgtatgccaactcctgatgcgccaatgtctaatcatcaagcatccaccatgagagagctgatttgcagttacttcaaggtcgatcatgcggatgggttgaaatatatacccatggatgagagatactctccgtcatcacttgccagattgtttaccatgggcatggctgggctgcacattaccactgagccatcttataagcgtgttccgattatgcacttagctgcggacttggactgtatgacgctggctctaccttacatgattacgcttgatggtgatactgtggttcctgtcgctccaacactgtcagcggaacagcttctggacgacggactcaaaggattagcatgcatggatatctcctatggatgtgaggtggacgcgaatagccggccggctggtgatcagagtatggactcttcacgctgcatcaacgagttgtattgcgaggagacagcagaagccatctgtgtgcttaagacatgccttgtgttaaattgcatgcagtttaaacttgagatggatgacctagcacataacgctgctgagctggacaagatacagatgatgatacccttcagtgagcgtgtttttaggatggcctcgtcctttgcgactattgatgcccagtgttttaggttttgcgtgatgatgaaggataaaaatctgaaaatagatatgcgtgaaacgacgagactgtggactcgttcagcatcagatgattctgtggccacgtcatctttaagtatttccctggaccggggtcgatgggtggcggctgacgccagtgatgctagactgctggtttttccgattcgcgtgtaatgggtgagtgagctgatgtggtcgccaagacatgtgccggtgtcttggtggtgggtgacgcctaatcatc

SEQ ID NO: 4 - DNA, 1196 bp, reovirus
gctatttttgcctcttcccagacgttgtcgcaatggaggtgtgcttgcccaacggtcatcaggtcgtggacttaattaacaacgcttttgaaggtcgtgtatcaatctacagcgcgcaagagggatgggacaaaacaatctcagcacagccagatatgatggtatgtggtggcgccgtcgtttgcatgcattgtctaggtgttgttggatctctacaacgcaagctgaagcatttgcctcaccatagatgtaatcaacagatccgtcatcaggattacgtcgatgtacagttcgcagaccgtgttactgctcactggaagcggggtatgctgtccttcgttgcgcagatgcacgagatgatgaatgacgtgtcgccagatgacctggatcgtgtgcgtactgagggaggttcactagtggagctgaaccggcttcaggttgacccaaattcaatgtttagatcaatacactcaagttggacagatcctttgcaggtggtggacgaccttgacactaagctggatcagtactggacagccttaaacctgatgatcgactcatccgacttgatacccaactttatgatgagagacccatcacacgcgttcaatggtgtgaaactgaagggagatgctcgtcaaacccaattctccaggacttttgattcgagatcgagtttggaatggggtgtgatggtttatgattactctgagctggatcatgatccatcgaagggccgtgcttacagaaaggaattggtgacgccagctcgagatttcggtcactttggattatcccattattctagggcgactaccccaatccttggaaagatgccggccgtattctcaggaatgttgactgggaactgtaaaatgtatccattcattaaaggaacggctaagctgaagacagtgcgcaagctagtggaggcagtcaatcatgcttggggtgtcgagaagattagatatgctcttgggccaggtggcatgacgggatggtacaataggactatgcaacaggcccccattgtgctaactcctgctgctctcacaatgttcccagataccatcaagtttggggatttgaattatccagtgatgattggcgatccgatgattcttggctaaacacccccatcttcacagcgccgggcttgaccaacctggtgtgacgtgggacaggcttcattcatc

SEQ ID NO: 5 - DNA, 2304 bp, reovirus
gctattcgcggtcatggcttacatcgcagttcctgcggtggtggattcacgttcgagtgaggctattggactgctagaatcgtttggagtagacgctggggctgacgcgaatgacgtttcatatcaagatcatgactatgtgttggatcagttacagtacatgttagatggatatgaggctggtgacgttatcgatgcactcgtccacaagaattggttacatcactctgtctattgcttgttgccacccaaaagtcaactattagagtattggaaaagtaatccttcagcgataccggacaacgttgatcgtcggcttcgtaaacgactaatgctaaagaaagatctcaggaaagatgatgaatacaatcagctagcgcgtgctttcaagatatcggatgtctacgcacctctcatctcatccacgacgtcaccgatgacaatgatacagaacttgaatcgaggcgagatcgtgtacaccacgacggacagggtaataggggctagaatcttgttatatgctcctagaaagtactatgcgtcaactctgtcatttactatgactaagtgcatcattccgtttggtaaagaggtgggtcgtgttcctcactctcgatttaatgttggcacatttccgtcaattgctaccccgaaatgttttgtcatgagtggggttgatattgagtccatcccaaatgaatttatcaagttgttttaccagcgcgtcaagagtgttcacgctaacatactaaatgacatatctcctcagatcgtctctgacatgataaacagaaagcgtctgcgcgttcatactccatcagatcgtcgagccgcgcagttgatgcatttgccttaccatgttaaacgaggagcgtctcacgtcgacgtttacaaggtggatgttgtagacatgttgttcgaggtagtggatgtggccgatgggttgcgcaacgtatctaggaaactaactatgcataccgttcctgtatgtattcttgaaatgttgggtattgagattgcggactattgcattcgtcaagaggatggaatgctcacagattggttcctacttttaaccatgctatctgatggcttgactgatagaaggacgcattgtcaatacttgattaatccgtcaagtgtgcctcctgatgtgatacttaacatctcaattactggatttataaatagacatacaatcgatgtcatgcctgacatatatgacttcgttaaacccattggcgctgtgctgcctaagggatcatttaaatcaacaattatgagagttcttgattcaatatcaatattaggaatccaaatcatgccgcgcgcgcatgtagttgactcagatgaggtgggcgagcaaatggagcctacgtttgagcaggcggttatggagatatacaaagggattgctggcgttgactcgctggatgatctcatcaagtgggtgttgaactcggatctcattccgcatgatgacaggcttggtcaattatttcaagcgtttttgcctctcgcaaaggacttattagctccaatggccagaaagttttatgataactcaatgagtgagggtagattgctaacattctctcatgccgacagtgagttgctgaacgcaaattattttggtcatttattgcgactaaaaataccatatattacagaggttaatctgatgattcgcaagaatcgtgagggtggagagctatttcagcttgtgttatcttatctatataaaatgtatgctactagcgcgcagcctaaatggtttggatcattattgcgattgttaatatgtccctggttacatatggagaaattaataggagaagcagacccggcatctacgtcggctgaaattgggtggcatatccctcgtgaacagctgatgcaagatggatggtgtggatgtgaagacggattcattccctatgttagcatacgtgcgccaagactggttatagaggagttgatggagaagaactggggccaatatcatgcccaagttattgtcactgatcagcttgtcgtaggcgaaccgcggagggtatctgctaaggctgtgatcaagggtaaccacttaccagttaagttagtttcacgatttgcatgtttcacattgacggcgaagtatgagatgaggctttcgtgcggccatagcactggacgtggagctgcatacagtgcgagactagctttccgatctgacttggcgtgatccgtgacatgcgtagtgtgacacctgctcctaggtcaatgggggtagggggcgggctaagactacgtacgcgcttcatc

SEQ ID NO: 6 - DNA, 2204 bp, reovirus
ggctaatctgctgaccgttactctgcaaagatggggaacgcttcctctatcgttcagacgatcaacgtcactggagatggcaatgtatttaaaccatcagctgaaacttcatctaccgctgtaccatcgttaagcttatcacctggaatgctgaatcccggaggggtaccatggattgctgttggagatgagacatctgtgacttcaccaggcgcattacgtcgaatgacgtcaaaggacatcccggacacggcaataatcaacacagacaattcatcaggcgccgtgccaagcgaatcagccttggtgccctacatcgatgagccgctggtagtggttacagagcatgctattaccaacttcaccaaagctgagatggcacttgaattcaatcgtgagttccttgacaagatgcgtgtgctgtcagtgtcaccaaaatattcggatcttctgacctatgttgactgctacgtcggtgtgtctgctcgtcaggctttaaacaattttcagaaacaagtgcctgtgattacacctactaggcagacgatgtatgtcgactcgatacaagcggccttgaaagctttagaaaagtgggagattgatctgagagtggctcaaacgttgctgcctacgaacgttccgattggagaagtctcttgtccaatgcagtcggtagtgaaactgctggatgatcagctgccagatgacagcctgatacggaggtatcccaaggaagccgccgtcgctttggctaaacgaaacgggggaatacaatggatggacgtatcagaaggcaccgtgatgaacgaggctgtcaacgctgttgcagctagtgcactggcaccttcagcatcagccccacccttagaagagaagtcaaagttaaccgaacaagcgatggatctcgtgaccgcggctgagcctgagataattgcctcactcgcgccagttcccgcacccgtgtttgccataccacctaaaccagcagattataatgtgcgtactctgaggatcgacgaggccacttggctgcgaatgattccaaaatcaatgaacacaccttttcaaatccaggtgactgataacacaggaactaattggcatctcaatttgaggggggggactcgtgtagtgaatctggaccaaatcgctccgatgcggtttgtattagatctagggggaaagagttataaagagacgagctgggatccaaacggcaagaaggtcggattcatcgtttttcaatcgaagataccattcgaactttggactgctgcttcacagatcggtcaagccacggtggttaactatgtccaactatacgctgaagacagctcatttaccgcgcagtctatcattgctactacctctttggcttataactatgagcctgagcagttgaataagactgaccctgagatgaattattatcttttggcgacctttatagactcagccgctataacgccaacgaatatgacacagcctgatgtttgggatgccttgctgacgatgtccccactatcagctggcgaggtgacagtgaagggtgcggtagtgagtgaagtagtccctgcagacttgataggtagctacactccagaatccctaaacgcctcacttccgaatgatgctgctagatgcatgatcgatagagcttcgaagatagccgaagcaatcaagattgatgatgatgctggaccagatgaatattccccaaactctgtaccaattcaaggtcagcttgctatctcgcaactcgaaactggatatggtgtgcgaatattcaaccctaaagggatcctttctaaaattgcatctagggcaatgcaggctttcattggtgacccgagcacaatcatcacgcaggcggcgccagtgttatcagacaagaataattggattgcattggcacagggagtgaaaactagtctgcgtactaaaagtctatcagcgggagtgaagactgcagtgagtaagctgagctcatctgagtctatccagaattggactcaaggattcttggataaagtgtcagcgcattttccagcaccaaagcccgattgtccgactagcggagatagtggtgaatcgtctaatcgccgagtgaagcgcgactcatacgcaggagtggtcaaacgtgggtacacacgttaggccgctcgccctggtgacgcggggttaagggatgcaggcaaatcatc

SEQ ID NO: 7 - DNA, 2241 bp, revovirus
gctaaagtgaccgtggtcatggcttcattcaagggattctccgccaacactgttccagtttctaaggccaagcgtgacatatcatctcttgccgctactcctggacttcgttcacaatccttcactccgtctgtggatatgtctcaatcgcgtgaattcctcacaaaggcaattgagcaagggtccatgtctataccttatcagcatgtgaatgtaccgaaagttgatcgtaaagttgttagcctggtagtgcgacctttctcttcaggtgctttctctatctctggagtgatttcgccagcccatgcctatctactagagtgtctaccccagcttgagcaggcgatggcttttgttgcttcacctgagtctttccaggcttccgacgtcgcgaagcgctttgccataaagccaggtatgagcctccaggatgccatcactgcctttattaactttgtgtccgcgatgctgaaaatgacggtgactcgtcaaaactttgacgttattgtggctgagatcgagaggcttgcttcaaccagcgtgtccgtcaggactgaagaagcgaaggttgctgatgaggagctaatgctattcgggttagatcatagagggccacagcagctggatgtttctgacgctaaagggataatgaaggctgctgatattcagacaactcatgatgtccatttggcaccaggcgttggtaatattgatcctgaaatctataacgaggggcggttcatgttcatgcagcacaagccacttgcggcggatcaatcgtatttcaccttggagactgcggattatttcaagatttatccaacatacgatgaacatgatggcaggatggctgaccaaaagcagtcgggattgatactgtgtactaaggacgaggtattggctgagcaaactatatttaaactggacgcccctgatgacaagactgttcatctgttggatcgcgatgacgaccacgttgttgccagatttactaaggtatttatagaggacgtggctcccgggcatcatgctgctcaaagatcgggacaacgctctgtgcttgatgacctatatgcgaatacgcaagtgatttccattacttctgctgctttaaagtgggtggtcaagcacggcgtatctgatggaatcgtgaacaggaagaatgtcaaagtgtgtgttggttttgaccccctgtacaccttgtctacacataacggggtgtccttatgtgccctgctgatggacgaaaaactctctgtgctgaacagtgcgtgtcgtatgacgttacgctcactcatgaagaccggacgcgacgttgatgcacacagagcttttcagcgagtcctctctcaaggatacacatcgctaatgtgctactatcatccttcacggaagttggcatatggtgaggtgctctttctagaacgatccaatgacgtgacagatgggatcaagcttcagttggacgcatctagacagtgtcatgaatgtcctgtgttgcagcagaaagtggttgagttagagaaacagattattatgcagaagtcaatccagtcagaccctaccccagtggcgctgcaaccattgttgtctcagttgcgtgagttgtctagtgaagttactaggctacagatggagttgagtcgagctcagtccctgaatgctcagttggaggcggatgtcaagtcagctcaatcatgtagcttggatatgtatctgagacaccacacttgcattaatggtcatgctaaagaagatgaattgcttgacgctgtgcgtgtcgcgccggatgtgaggagagaaatcatggaaaagaggagtgaagtgagacaaggttggtgcgaacgtatttctaaggaagcagctgccaaatgtcaaactgttattgatgacctgactttgatgaatggaaagcaagcacaagagataacagaattacgtgattcggctgaaaaatatgagaaacagattgcagagctggtgagtaccatcacccaaaaccagataacgtatcagcaagagctacaagccttggtagcgaaaaatgtggaattggacgcgttgaatcagcgtcaggctaagtctttgcgtattactccctctcttctatcagccactcctatcgattcagttgatgatgttgctgacttaattgatttctctgttccaactgatgagttgtaaataatccgtgatgcagtgttgccctaatcccttaagccttcccgacccccattcatc

SEQ ID NO: 8 - DNA, 3854 bp, reovirus
gctacacgttccacgacaatgtcatccatgatactgactcagtttggaccgttcattgagagcatttcaggtatcactgatcaatcgaatgacgtgtttgaagatgcagcaaaagcattctctatgtttactcgcagcgatgtctacaaggcgctggatgaaatacctttctctgatgatgcgatgcttccaatccctccaactatatatacgaaaccatctcacgattcatattattacattgatgctctaaaccgtgtgcgtcgcaaaacatatcagggccctgatgacgtgtacgtacctaattgttctattgttgaattgctggagccacatgagactctgacatcttatgggcggttgtccgaggccatcgagaatcgtgccaaggatggggacagccaagccagaatcgccacaacgtatggtagaatcgctgaatctcaagctcgacagattaaggctccattggagaagtttgtgttggcactattagtggccgaagcaggggggtctttatatgatccagttttgcagaagtatgatgagattccagatctatcgcataattgccctttatggtgttttagagagatctgtcgtcacatatctggtccattaccagatcgggcaccttatctttacttatctgcaggggttttctggttaatgtcaccacgaatgacgtctgcaatccctccgctactatccgatcttgttaatttagctattttgcaacaaactgcgggtttagatccatcattagtgaaattgggagtacagatatgccttcatgcagcagctagctcaagttatgcatggtttatcttaaagactaagtctatttttcctcaaaacacgttgcacagtatgtatgaatctctagaagggggatactgtcctaatcttgaatggttagagcctagatcagactataagttcatgtacatgggagtcatgccattgtccgctaagtatgctaggtcggcgccgtccaatgataagaaagcgcgggaacttggcgagaaatatggactgagctcagtcgtcggtgagcttcgtaaacggacaaagacgtatgttaaacatgactttgcttcagtgaggtacattcgtgacgctatggcatgtactagcggtattttcttggtaagaacacccaccgaaacggtattgcaagaatatacgcagagtccggagattaaggttcccattccccagaaagactggacaggcccaataggtgaaatcagaattctaaaagatacaacaagttccatcgcgcgttacttatatagaacatggtacttggcagcggcgagaatggcggctcaaccacgtacgtgggatccattgtttcaagcgattatgagatctcaatacgtgacagctaggggtggatctggcgcagcactccgcgaatctttgtatgcaatcaatgtgtcgttacctgatttcaagggcttaccagtgaaggcagcaactaagatattccaggcggcacaattagcgaacttgccgttctcccacacatcagtggctatactagctgacacttcaatgggattgcgaaatcaggtgcagaggcggccacgatccattatgccattaaatgtgccccagcagcaggtttcggcgccccatacattgacagcggattacattaactaccacatgaatctatcaaccacgtctggtagtgcggtcattgagaaggtgattcctttaggtgtatacgcttcgagccctcctaaccagtcgatcaacattgacatatctgcgtgtgacgctagtattacttgggatttctttctgtcagtgattatggcggctatacacgaaggtgtcgctagtagctccattggaaaaccatttatgggggttcctgcatccattgtaaatgatgagtctgtcgttggagtgagagctgctaggccgatatcgggaatgcagaacatgattcagcatctatcgaaactatataaacgtggattttcatatagagtaaacgattctttttctccaggtaacgattttactcatatgactaccactttcccgtcaggttcaacagccacctctactgagcatactgctaataatagtacgatgatggaaactttcctgacagtatggggacccgaacatactgacgaccctgacgtcttacgtttaatgaagtctttaactattcaaaggaattacgtatgtcaaggtgatgatggattaatgattatcgatgggactactgctggtaaggtgaacagtgaaactattcagaagatgctagaattaatctcaaaatatggtgaggaattcggatggaaatatgacatagcgtacgatgggactgccgaatacttaaagctatacttcatatttggctgtcgaattccaaatcttagtcgccatccaatcgtggggaaagaacgggcgaattcttcagcagaggagccatggccagcaattctagatcagattatgggtgtcttctttaatggtgttcatgatgggttacagtggcagcggtggatacgttattcatgggctctatgctgtgctttctcacgtcaaagaacaatgattggtgagagcgtgggttaccttcaatatcctatgtggtcttttgtctactggggattaccactggttaaagcgtttgggtcagacccatggatattttcttggtacatgcctactggagatctgggaatgtatagttggattagcttgatacgccctctgatgacaagatggatggtggctaatggttacgtaactgacagatgctcacccgtattcgggaacgcagattatcgcaggtgtttcaatgaacttaaactatatcaaggttattatatggcacaattgcccaggaatcctaagaagtctggacgagcggcccctcgggaggtaagagaacaattcactcaggcattatccgactatctactgcaaaatccagagctgaagtcacgtgtgctacgtggtcgtagtgagtgggagaaatatggagcggggataattcacaatcctccgtcattattcgatgtgccccataaatggtatcagggtgcgcaagaggcagcaatcgctacgagagaagagctggcagaaatggatgagacattaatgcgcgctcgaaggcacagatattcgagcttttcaaagttattagaggcgtatctgctcgtgaaatggcgaatgtgcgaggcccgcgaaccgtcggtggatttgcgattaccattatgtgcgggtattgacccattaaactcagatccttttctcaagatggtaagcgttggaccaatgctccagagtacgagaaagtactttgctcagacactattcatggcaaagacggtgtcgggtcttgacgttaacgcgattgatagcgcgttattacgactgcgaacattaggtgctgataagaaagcattaacggcgcagttattaatggtggggcttcaggagtcagaagcggacgcattggccgggaagataatgctacaggatgtgaatactgtgcaattagccagagtggttaacttagctgtgccagatacttggatgtcgttagactttgactctatgttcaaacaccacgtcaagctgcttcccaaagatggacgtcatctaaatactgatattcctcctcgaatgggatggttacgggccattttacgattcttaggtgccggaatggtaatgactgcgactggagttgctgtcgacatctatctggaggatatacatggcggtggtcggtcacttggacagagattcatgacttggatgcgacaggaaggacggtcagcgtgagtctaccatgggtcgtggtgcgtcaactcatc

SEQ ID NO: 9 - DNA, 3916 bp, reovirus
gctaaatggcgcgatggcgaacgtttggggggtgagacttgcagactcgttatcttcacccactattgagacacgaacgcgtcagtataccttacacgatctttgctcagacctagatgctaatccggggagggaaccgtggaaacctctgcgtaatcagcgtactaataatattgtggctgtgcaattattcagaccattgcagggtttagttttagatacccagctttatggatttccaggagcatttgatgactgggagcgattcatgagagagaagctgcgtgtgctaaagtatgaagtattgcgcatctatccaatcagcaactatagcaatgaacatgtcaacgtcttcgtggccaatgctttggtgggcgctttcctgtcgaatcaagctttctatgacctgctaccgttgttgataattaatgacactatgattggtgatctacttggcacgggggcatcgctatcacagttctttcaatctcatggagatgtgctggaagtcgcagctggtcgtaagtatctgcagatggaaaactactccaacgatgacgatgatcctccattatttgcgaaagacctgtcagattatgctaaagcattctacagtgacacatatgaagtgttggacaggttcttttggacgcatgactcttcagcgggggtcttagtgcattatgataagccaacgaatggtcatcactatctgctgggtactttgactcagatggtcagtgcacctccttatattattaacgctactgacgcaatgttgcttgaatcctgtctagaacagttctcagctaatgtgcgtgcgagacctgcgcaacccgttacacgcttagaccaatgctatcatttaagatggggagcacaatatgtaggagaagattcactgacatatcggttgggggtgttatccttgctggctaccaatggatatcaattagctagaccgattccaagacagttgacgaatcgatggttgtcgagctttgtgagtcaaattatgtctgacggcgtcaacgagactccactgtggccccaagaaaggtatgtgcagatcgcttatgattcaccatccgttgttgatggggctacgcaatatggctatgtcaggaagaatcaactcagactcggcatgagaatatcggcgctgcaatcgctgagtgatacgccctcgccggtacagtggcttccacaatacaccatcgaccaggcagcgatggacgaaggcgatctgatggttagtcggcttacgcaactcccgttacgtcctgattatggtaatatctgggtcggcgatgcgctatcctattatgtggactacaatcggagtcatcgagtcgtgctttcatcggaacttcctcagcttccggacacatattttgatggcgatgaacagtatgggcgcagcctgttctcactagctcgtaagattggtgaccgctcgttagtgaaagatacggctgtcttgaagcacgcttaccaagccatcgatccaaatactggtaaggagtatctgagatctcggcaatctgtcgcatattttggtgcatcagcgggtcattctggtgccgaccagccgttagtcatagagccctggattcaagggaaaatcagtggtgtgccgccaccctcctcagtgcgacagttcggctatgatgttgcccgtggcgcgatcgtcgatctggcgagaccatttccttctggagattatcaatttgtctattcggatgttgaccaggtggtcgatggccatgacgatctgagtatatcatctggactggtggagagccttttgtcttcatgcatgcacgccacagcacccgggggctcatttgttgttaagataaattttccgactagacccgtatggcactacatcgaacagaagatcttgcccaatattacgtcatacatgttgatcaagcctttcgtcaccaacaacgtcgaattgttcttcgtcgctttcggtgtgcatcaacactcatcacttacttggacatctggagtgtacttcttcttggtggaccatttttatcgttatgagactttatctacgatctcacgacaattgccgtcttttgggtatgttgatgatgggtcttccgtgactggtatcgagacaattagtattgagaaccctggcttctcgaatatgacccaggccgctcgcattggtatctcaggattgtgtgctaatgtaggtaacgcgcgtaagtccattgccatttacgaatctcatggggccagagtattaactatcacatcaaggagatctccggcatcagctagaagaaagtctaggttgcgatatttgccattaatagaccctaggtcgttagaggtacaggcgcgcactattctgccagctgatccagtgttatttgaaaacgtgagcggagcgtcaccccatgtttgtctgacaatgatgtacaacttcgaagtgtcgtcagcggtatatgatggagacgttgtgctagatcttgggacgggaccagaggctaaaatccttgaactgatacccgcaacctctccagtcacatgcgtggacatacggcctacagcgcagcctagtggatgttggaacgttcgtaccacgttccttgagttagattatttgagcgatggatggatcactggggtgcgtggggacatagttacttgtatgttatctttgggggccgctgccgctggaaaatcaatgacttttgacgctgcgtttcagcaattaatcaaagtattatccaagagtacggctaatgttgtgctggtgcaggttaactgccctacagacgtggtgaggagcattaagggctacctagagatagattcgactaacaagaggtataggttccccaaatttggtcgagacgagccgtactctgacatggatgcgctggagaaaatatgtcgtaccgcctggccaaactgctcaattacctgggttccattgtcatacgacttgcggtggactagactggcattattagagtccacgacattgagtagcgcgtcgattagaattgctgagctgatgtataaatacatgcctattatgaggattgatattcatggactacccatggaaaagcgaggtaacttcatagtggggcagaactgctcattagtaatccctggttttaatgcgcaggatgtctttaactgttatttcaattccgccctcgctttctcgactgaagatgtcaatgctgcgatgattccccaagtgtctgcgcagtttgatgcgactaagggtgagtggacgttggatatggtcttctccgacgcaggaatctataccatgcaggctctagtgggatctaatgctaatccagtctctttgggttcctttgtagttgattctccagatgtagatataactgacgcttggccagctcagttagactttacgatcgcgggaactgatgtcgatataacagttaatccttattaccgtctgatgacctttgtaaggatcgatggacagtggcagattgccaatccagacaaatttcaattcttttcgtcggcgtctgggacgttagtgatgaacgtcaaattagatatcgcagataaatatctactatactatatacgagatgtccagtctcgagatgttggcttttacattcagcatccacttcaacttttgaatacgatcacattgccaaccaacgaggacctttttctgagcgcacctgacatgcgagagtgggcagttaaggaaagcggtaacacgatatgtatactcaatagtcaagggtttgtgctacctcaagattgggatgtgttaacagataccataagttggtccccatcgatacccacatacattgtgccaccgggtgattataccttgactcctctgtaactcactgtccctcgtgagcgcgcctaattcatc

SEQ ID NO: 10 - DNA, 3901 bp, reovirus
gctaatcgtcaggatgaagcggattccaaggaagacaaagggcaaatccagcggaaagggcaatgactcaacagagagagcggacgatggctcgagccaattaagagacaagcaaaacaataaggctggccccgccactacggagcctggcacatccaaccgagagcaatacaaagctcgaccaggtattgcatctgtgcagagggccactgaaagtgcagaaatgcccatgaagaataatgacgaagggacgccagataagaaaggaaatactaagggcgacctagttaatgagcatagtgaggctaaagacgaggcggatgaagcgacgaagaagcaggcaaaggatacagacaaaagtaaagcgcaagtcacatattcagacactggtatcaataatgctaatgaactgtcaagatctgggaatgtggataatgagggtggaagtaatcagaagccgatgtctaccagaatagctgaggcaacgtctgctatagtgtcgaaacatcctgcgcgtgttgggctgccacctaccgctagcagtggtcatgggtatcagtgccatgtctgttctgcagtcctgtttagtcctttagacctagatgcccacgtcgcctcacatggtttgcatggtaacatgacattaacatcgagtgatatccagcgacatataactgagttcatcagctcatggcaaaatcatcctattgttcaagtttcggctgatgtcgaaaataagaaaactgctcaattgcttcacgctgacactcctcgactcgtcacttgggatgctggtttgtgtacttcattcaaaatcgtcccgattgtgccagctcaggtgccgcaggatgtactggcctatacgtttttcacctcttcatacgctatccaatcaccgtttccagaggcggcagtgtctaggattgtggtgcatacgagatgggcatctaatgttgactttgaccgagactcgtctgtcatcatggcgccacctacagaaaacaatatccatttgtttaaacagttactaaatactgaaaccctgtctgtaaggggggctaatccgctaatgttcagggcgaatgtgttgcatatgttgctagagttcgtattagataacttgtatctgaacagacatacgggattctctcaagaccacacgccatttactgagggtgctaatttgcgttcacttcctggccccgatgctgagaaatggtactcgattatgtatccaacgcgcatgggaacgccgaatgtatccaaaatatgtaatttcgtcgcctcttgtgtgcgaaatcgggttggacggtttgatcgagcacagatgatgaacggagctatgtcagagtgggtggatgtcttcgagacttcagacgcgctaaccgtctccattcgaggtcgatggatggctagactagctcgcatgaacataaatccaacagagatcgaatgggcattgactgaatgtgcacaaggatatgtgactgtcacaagtccttacgctcctagcgtaaatagattgatgccctatcgtatctccaacgctgagcggcaaatatcacagataatcaggatcatgaacattggcaataacgcgacggtgatacaacctgttctgcaagatatttcggtactccttcaacgcatatcaccactccaaatagatccaactattatttccaacactatgtcaacagtctcggagtctactactcagaccctcagccccgcgtcctcaattttgggtaaactacgaccaagcaactcagatttttctagttttagagtcgcgttggctggatggctttataatggggttgtgacgacggtgattgatgatagttcatatccaaaagacggcggcagcgtgacctcacttgaaaatctgtgggatttcttcatccttgcgcttgctctaccactgacaactgacccctgtgcacctgtgaaagcattcatgaccctagccaacatgatggttggtttcgagacaatccctatggataatcagatctatactcaatcgagacgcgcgagtgctttctcaacgcctcacacgtggccacgatgctttatgaacatccagttaatttctccaatcgacgctcccatcttgcgacagtgggctgaaattattcatagatactggcctaacccttcacagattcgttatggtgcaccgaacgttttcggctcggcaaatttgttcactccacctgaggtgctgttattgccaatcgatcatcaaccagctaatgtaacaacgccaacgctggacttcaccaatgagttaactaattggcgcgctcgtgtctgtgagcttatgaagaatctcgttgataaccaaagatatcaacctggatggacacaaagtctagtctcgtcaatgcgcggaacgctagacaaattgaagttgattaaatcgatgacaccaatgtatctgcaacagctggctccggtagagttagcagtgatagctcccatgttgccttttccacctttccaggtgccatacgtccgtctcgatcgtgacagagttccaacaatggttggagtaacacgacattcacgagatactattactcagccggcgctatcgctgtcgacaaccaatactactgttggcgtgccactagctctagacgcgagggctatcaccgttgcgctgttgtcagggaaatatccgccggatttggtgacaaatgtatggtacgctgatgccatttacccaatgtatgcagacacggaggtgttctctaatcttcagagagacatgattacctgcgaggccgtgcagacattagtgactctggtggcgcaaatatcagagacccagtatcctgtagataggtatcttgattggatcccatcactgagagcatcggcggcgacggcggcgacatttgctgagtgggttaatacttcaatgaagacggcgtttgatttgtctgatatgctgttagagcctctcctaagcggtgatccgaggatgactcaactagcgattcagtatcagcagtacaatggcagaacgtttaatatcatacctgaaatgccaggttcagtaattgctgactgcgttcaattaacagcagaagtctttaatcacgaatataacctgtttgggattgcgcggggtgatatcatcattggccgtgttcagtcgacacatttgtggtcaccgctggctcctccacctgacctggtgtttgatcgtgatacccctggtgttcacatcttcggacgagattgccgtatatcgtttggaatgaatggcgccgcgccaatgattagagatgagactggactgatggtgccttttgaaggaaattggattttcccactggcgctttggcaaatgaatacacgatattttaatcaacagttcgacgcgtggattaagacaggagagttgcgaatccgcattgagatgggcgcgtatccatatatgttgcattactatgatccacgtcagtacgctaatgcatggaatttaacatccgcctggcttgaagaaattacgccgacgagcatcccatccgtgcctttcatggtgcccatttcaagtgatcatgacatttcctctgccccagctgtccaatatatcatttcaactgaatataatgatcggtctctgttctgcactaactcatcatctccccaaaccatcgctggaccagacaaacacattccagttgagagatataacattctgaccaaccccgacgctccacccacgcagatacaactgcctgaagtcgttgacttgtacaacgtcgtcacacgctatgcgtatgagactccgcctattaccgctgttgttatgggtgttccttgatcctcatcctcccaacaggtgctagagcattgcgctcaatgctagttgggccgattcatc

SEQ ID NO: 11 - protein, 455 aa, reovirus
MDPRLREEVVRLIIALTSDNGASLSKGLESRVSALEKTSQIHSDTILRITQGLDDANKRIIALEQSRDDLVASVSDAQLAISRLESSIGALQTVVNGLDSSVTQLGARVGQLETGLAELRVDHDNLVARVDTAERNIGSLTTELSTLTLRVTSIQADFESRISTLERTAVTSAGAPLSIRNNRMTMGLNDGLTLSGNNLAIRLPGNTGLNIQNGGLQFRFNTDQFQIVNNNLTLKTTVFDSINSRIGATEQSYVASAVTPLRLNSSTKVLDMLIDSSTLEINSSGQLTVRSTSPNLRYPIADVSGGIGMSPNYRFRQSMWIGIVSYSGSGLNWRVQVNSDIFIVDDYIHICLPAFDGFSIADGGDLSLNFVTGLLPPLLTGDTEPAFHNDVVTYGAQTVAIGLSSGGAPQYMSKNLWVEQWQDGVLRLRVEGGGSITHSNSKWPAMTVSYPRSFT

SEQ ID NO: 12 - protein, 418 aa, reovirus
MARAAFLFKTVGFGGLQNVPINDELSSHLLRAGNSPWQLTQFLDWISLGRGLATSALVPTAGSRYYQMSCLLSGTLQIPFRPNHRWGDIRFLRLVWSAPTLDGLVVAPPQVLAQPALQAQADRVYDCDDYPFLARDPRFKHRVYQQLSAVTLLNLTGFGPISYVRVDEDMWSGDVNQLLMNYFGHTFAEIAYTLCQASANRPWEYDGTYARMTQIVLSLFWLSYVGVIHQQNTYRTFYFQCNRRGDAAEVWILSCSLNHSAQIRPGNRSLFVMPTSPDWNMDVNLILSSTLTGCLCSGSQLPLIDNNSVPAVSRNIHGWTGRAGNQLHGFQVRRMVTEFCDRLRRDGVMTQAQQNQVEALADQTQQFKRDKLETWAREDDQYNQAHPNSTMFRTKPFTNAQWGRGNTGATSAAIAALI

SEQ ID NO: 13 - protein, 254 aa, reovirus
MASSLRAAISKIKRDDVGQQVCPNYVMLRSSVTTKVVRNVVEYQIRTGGFFSCLAMLRPLQYAKRERLLGQRNLERISTRDILQTRDLHSLCMPTPDAPMSNHQASTMRELICSYFKVDHADGLKYIPMDERYSPSSLARLFTMGMAGLHITTEPSYKRVPIMHLAADLDCMTLALPYMITLDGDTVVPVAPTLSAEQLLDDGLKGLACMDMDVRWTRIAGRLVIRVWTLHAASTSCIARRQQKPSVCLRHALC

SEQ ID NO: 14 - protein, 366 aa, reovirus
MASSLRAAISKIKRDDVGQQVCPNYVMLRSSVTTKVVRNVVEYQIRTGGFFSCLAMLRPLQYAKRERLLGQRNLERISTRDILQTRDLHSLCMPTPDAPMSNHQASTMRELICSYFKVDHADGLKYIPMDERYSPSSLARLFTMGMAGLHITTEPSYKRVPIMHLAADLDCMTLALPYMITLDGDTVVPVAPTLSAEQLLDDGLKGLACMDISYGCEVDANSRPAGDQSMDSSRCINELYCEETAEAICVLKTCLVLNCMQFKLEMDDLAHNAAELDKIQMMIPFSERVFRMASSFATIDAQCFRFCVMMKDKNLKIDMRETTRLWTRSASDDSVATSSLSISLDRGRWVAADASDARLLVFPIRV

SEQ ID NO: 15 - protein, 365 aa, reovirus
MEVCLPNGHQVVDLINNAFEGRVSIYSAQEGWDKTISAQPDMMVCGGAVVCMHCLGVVGSLQRKLKHLPHHRCNQQIRHQDYVDVQFADRVTAHWKRGMLSFVAQMHEMMNDVSPDDLDRVRTEGGSLVELNRLQVDPNSMFRSIHSSWTDPLQVVDDLDTKLDQYWTALNLMIDSSDLIPNFMMRDPSHAFNGVKLKGDARQTQFSRTFDSRSSLEWGVMVYDYSELDHDPSKGRAYRKELVTPARDFGHFGLSHYSRATTPILGKMPAVFSGMLTGNCKMYPFIKGTAKLKTVRKLVEAVNHAWGVEKIRYALGPGGMTGWYNRTMQQAPIVLTPAALTMFPDTIKFGDLNYPVMIGDPMILG

SEQ ID NO: 16 - protein, 736 aa, reovirus
MAYIAVPAVVDSRSSEAIGLLESFGVDAGADANDVSYQDHDYVLDQLQYMLDGYEAGDVIDALVHKNWLHHSVYCLLPPKSQLLEYWKSNPSAIPDNVDRRLRKRLMLKKDLRKDDEYNQLARAFKISDVYAPLISSTTSPMTMIQNLNRGEIVYTTTDRVIGARILLYAPRKYYASTLSFTMTKCIIPFGKEVGRVPHSRFNVGTFPSIATPKCFVMSGVDIESIPNEFIKLFYQRVKSVHANILNDISPQIVSDMINRKRLRVHTPSDRRAAQLMHLPYHVKRGASHVDVYKVDVVDMLFEVVDVADGLRNVSRKLTMHTVPVCILEMLGIEIADYCIRQEDGMLTDWFLLLTMLSDGLTDRRTHCQYLINPSSVPPDVILNISITGFINRHTIDVMPDIYDFVKPIGAVLPKGSFKSTIMRVLDSISILGIQIMPRAHVVDSDEVGEQMEPTFEQAVMEIYKGIAGVDSLDDLIKWVLNSDLIPHDDRLGQLFQAFLPLAKDLLAPMARKFYDNSMSEGRLLTFSHADSELLNANYFGHLLRLKIPYITEVNLMIRKNREGGELFQLVLSYLYKMYATSAQPKWFGSLLRLLICPWLHMEKLIGEADPASTSAEIGWHIPREQLMQDGWCGCEDGFIPYVSIRAPRLVIEELMEKNWGQYHAQVIVTDQLVVGEPRRVSAKAVIKGNHLPVKLVSRFACFTLTAKYEMRLSCGHSTGRGAAYSARLAFRSDLA

SEQ ID NO: 17 - protein, 708 aa, reovirus
MGNASSIVQTINVTGDGNVFKPSAETSSTAVPSLSLSPGMLNPGGVPWIAVGDETSVTSPGALRRMTSKDIPDTAIINTDNSSGAVPSESALVPYIDEPLVVVTEHAITNFTKAEMALEFNREFLDKMRVLSVSPKYSDLLTYVDCYVGVSARQALNNFQKQVPVITPTRQTMYVDSIQAALKALEKWEIDLRVAQTLLPTNVPIGEVSCPMQSVVKLLDDQLPDDSLIRRYPKEAAVALAKRNGGIQWMDVSEGTVMNEAVNAVAASALAPSASAPPLEEKSKLTEQAMDLVTAAEPEIIASLAPVPAPVFAIPPKPADYNVRTLRIDEATWLRMIPKSMNTPFQIQVTDNTGTNWHLNLRGGTRVVNLDQIAPMRFVLDLGGKSYKETSWDPNGKKVGFIVFQSKIPFELWTAASQIGQATVVNYVQLYAEDSSFTAQSIIATTSLAYNYEPEQLNKTDPEMNYYLLATFIDSAAITPTNMTQPDVWDALLTMSPLSAGEVTVKGAVVSEVVPADLIGSYTPESLNASLPNDAARCMIDRASKIAEAIKIDDDAGPDEYSPNSVPIQGQLAISQLETGYGVRIFNPKGILSKIASRAMQAFIGDPSTIITQAAPVLSDKNNWIALAQGVKTSLRTKSLSAGVKTAVSKLSSSESIQNWTQGFLDKVSAHFPAPKPDCPTSGDSGESSNRRVKRDSYAGVVKRGYTR

SEQ ID NO: 18 - protein, 721 aa, reovirus
MASFKGFSANTVPVSKAKRDISSLAATPGLRSQSFTPSVDMSQSREFLTKAIEQGSMSIPYQHVNVPKVDRKVVSLVVRPFSSGAFSISGVISPAHAYLLECLPQLEQAMAFVASPESFQASDVAKRFAIKPGMSLQDAITAFINFVSAMLKMTVTRQNFDVIVAEIERLASTSVSVRTEEAKVADEELMLFGLDHRGPQQLDVSDAKGIMKAADIQTTHDVHLAPGVGNIDPEIYNEGRFMFMQHKPLAADQSYFTLETADYFKIYPTYDEHDGRMADQKQSGLILCTKDEVLAEQTIFKLDAPDDKTVHLLDRDDDHVVARFTKVFIEDVAPGHHAAQRSGQRSVLDDLYANTQVISITSAALKWVVKHGVSDGIVNRKNVKVCVGFDPLYTLSTHNGVSLCALLMDEKLSVLNSACRMTLRSLMKTGRDVDAHRAFQRVLSQGYTSLMCYYHPSRKLAYGEVLFLERSNDVTDGIKLQLDASRQCHECPVLQQKVVELEKQIIMQKSIQSDPTPVALQPLLSQLRELSSEVTRLQMELSRAQSLNAQLEADVKSAQSCSLDMYLRHHTCINGHAKEDELLDAVRVAPDVRREIMEKRSEVRQGWCERISKEAAAKCQTVIDDLTLMNGKQAQEITELRDSAEKYEKQIAELVSTITQNQITYQQELQALVAKNVELDALNQRQAKSLRITPSLLSATPIDSVDDVADLIDFSVPTDEL

SEQ ID NO: 19 - protein, 1267 aa, reovirus
MSSMILTQFGPFIESISGITDQSNDVFEDAAKAFSMFTRSDVYKALDEIPFSDDAMLPIPPTIYTKPSHDSYYYIDALNRVRRKTYQGPDDVYVPNCSIVELLEPHETLTSYGRLSEAIENRAKDGDSQARIATTYGRIAESQARQIKAPLEKFVLALLVAEAGGSLYDPVLQKYDEIPDLSHNCPLWCFREICRHISGPLPDRAPYLYLSAGVFWLMSPRMTSAIPPLLSDLVNLAILQQTAGLDPSLVKLGVQICLHAAASSSYAWFILKTKSIFPQNTLHSMYESLEGGYCPNLEWLEPRSDYKFMYMGVMPLSAKYARSAPSNDKKARELGEKYGLSSVVGELRKRTKTYVKHDFASVRYIRDAMACTSGIFLVRTPTETVLQEYTQSPEIKVPIPQKDWTGPIGEIRILKDTTSSIARYLYRTWYLAAARMAAQPRTWDPLFQAIMRSQYVTARGGSGAALRESLYAINVSLPDFKGLPVKAATKIFQAAQLANLPFSHTSVAILADTSMGLRNQVQRRPRSIMPLNVPQQQVSAPHTLTADYINYHMNLSTTSGSAVIEKVIPLGVYASSPPNQSINIDISACDASITWDFFLSVIMAAIHEGVASSSIGKPFMGVPASIVNDESVVGVRAARPISGMQNMIQHLSKLYKRGFSYRVNDSFSPGNDFTHMTTTFPSGSTATSTEHTANNSTMMETFLTVWGPEHTDDPDVLRLMKSLTIQRNYVCQGDDGLMIIDGTTAGKVNSETIQKMLELISKYGEEFGWKYDIAYDGTAEYLKLYFIFGCRIPNLSRHPIVGKERANSSAEEPWPAILDQIMGVFFNGVHDGLQWQRWIRYSWALCCAFSRQRTMIGESVGYLQYPMWSFVYWGLPLVKAFGSDPWIFSWYMPTGDLGMYSWISLIRPLMTRWMVANGYVTDRCSPVFGNADYRRCFNELKLYQGYYMAQLPRNPKKSGRAAPREVREQFTQALSDYLLQNPELKSRVLRGRSEWEKYGAGIIHNPPSLFDVPHKWYQGAQEAAIATREELAEMDETLMRARRHRYSSFSKLLEAYLLVKWRMCEAREPSVDLRLPLCAGIDPLNSDPFLKMVSVGPMLQSTRKYFAQTLFMAKTVSGLDVNAIDSALLRLRTLGADKKALTAQLLMVGLQESEADALAGKIMLQDVNTVQLARVVNLAVPDTWMSLDFDSMFKHHVKLLPKDGRHLNTDIPPRMGWLRAILRFLGAGMVMTATGVAVDIYLEDIHGGGRSLGQRFMTWMRQEGRSA

SEQ ID NO: 20 - protein, 1289 aa, reovirus
MANVWGVRLADSLSSPTIETRTRQYTLHDLCSDLDANPGREPWKPLRNQRTNNIVAVQLFRPLQGLVLDTQLYGFPGAFDDWERFMREKLRVLKYEVLRIYPISNYSNEHVNVFVANALVGAFLSNQAFYDLLPLLIINDTMIGDLLGTGASLSQFFQSHGDVLEVAAGRKYLQMENYSNDDDDPPLFAKDLSDYAKAFYSDTYEVLDRFFWTHDSSAGVLVHYDKPTNGHHYLLGTLTQMVSAPPYIINATDAMLLESCLEQFSANVRARPAQPVTRLDQCYHLRWGAQYVGEDSLTYRLGVLSLLATNGYQLARPIPRQLTNRWLSSFVSQIMSDGVNETPLWPQERYVQIAYDSPSVVDGATQYGYVRKNQLRLGMRISALQSLSDTPSPVQWLPQYTIDQAAMDEGDLMVSRLTQLPLRPDYGNIWVGDALSYYVDYNRSHRVVLSSELPQLPDTYFDGDEQYGRSLFSLARKIGDRSLVKDTAVLKHAYQAIDPNTGKEYLRSRQSVAYFGASAGHSGADQPLVIEPWIQGKISGVPPPSSVRQFGYDVARGAIVDLARPFPSGDYQFVYSDVDQVVDGHDDLSISSGLVESLLSSCMHATAPGGSFVVKINFPTRPVWHYIEQKILPNITSYMLIKPFVTNNVELFFVAFGVHQHSSLTWTSGVYFFLVDHFYRYETLSTISRQLPSFGYVDDGSSVTGIETISIENPGFSNMTQAARIGISGLCANVGNARKSIAIYESHGARVLTITSRRSPASARRKSRLRYLPLIDPRSLEVQARTILPADPVLFENVSGASPHVCLTMMYNFEVSSAVYDGDVVLDLGTGPEAKILELIPATSPVTCVDIRPTAQPSGCWNVRTTFLELDYLSDGWITGVRGDIVTCMLSLGAAAAGKSMTFDAAFQQLIKVLSKSTANVVLVQVNCPTDVVRSIKGYLEIDSTNKRYRFPKFGRDEPYSDMDALEKICRTAWPNCSITWVPLSYDLRWTRLALLESTTLSSASIRIAELMYKYMPIMRIDIHGLPMEKRGNFIVGQNCSLVIPGFNAQDVFNCYFNSALAFSTEDVNAAMIPQVSAQFDATKGEWTLDMVFSDAGIYTMQALVGSNANPVSLGSFVVDSPDVDITDAWPAQLDFTIAGTDVDITVNPYYRLMTFVRIDGQWQIANPDKFQFFSSASGTLVMNVKLDIADKYLLYYIRDVQSRDVGFYIQHPLQLLNTITLPTNEDLFLSAPDMREWAVKESGNTICILNSQGFVLPQDWDVLTDTISWSPSIPTYIVPPGDYTLTPL

SEQ ID NO: 21 - protein, 1275 aa, reovirus
MKRIPRKTKGKSSGKGNDSTERADDGSSQLRDKQNNKAGPATTEPGTSNREQYKARPGIASVQRATESAEMPMKNNDEGTPDKKGNTKGDLVNEHSEAKDEADEATKKQAKDTDKSKAQVTYSDTGINNANELSRSGNVDNEGGSNQKPMSTRIAEATSAIVSKHPARVGLPPTASSGHGYQCHVCSAVLFSPLDLDAHVASHGLHGNMTLTSSDIQRHITEFISSWQNHPIVQVSADVENKKTAQLLHADTPRLVTWDAGLCTSFKIVPIVPAQVPQDVLAYTFFTSSYAIQSPFPEAAVSRIVVHTRWASNVDFDRDSSVIMAPPTENNIHLFKQLLNTETLSVRGANPLMFRANVLHMLLEFVLDNLYLNRHTGFSQDHTPFTEGANLRSLPGPDAEKWYSIMYPTRMGTPNVSKICNFVASCVRNRVGRFDRAQMMNGAMSEWVDVFETSDALTVSIRGRWMARLARMNINPTEIEWALTECAQGYVTVTSPYAPSVNRLMPYRISNAERQISQIIRIMNIGNNATVIQPVLQDISVLLQRISPLQIDPTIISNTMSTVSESTTQTLSPASSILGKLRPSNSDFSSFRVALAGWLYNGVVTTVIDDSSYPKDGGSVTSLENLWDFFILALALPLTTDPCAPVKAFMTLANMMVGFETIPMDNQIYTQSRRASAFSTPHTWPRCFMNIQLISPIDAPILRQWAEIIHRYWPNPSQIRYGAPNVFGSANLFTPPEVLLLPIDHQPANVTTPTLDFTNELTNWRARVCELMKNLVDNQRYQPGWTQSLVSSMRGTLDKLKLIKSMTPMYLQQLAPVELAVIAPMLPFPPFQVPYVRLDRDRVPTMVGVTRHSRDTITQPALSLSTTNTTVGVPLALDARAITVALLSGKYPPDLVTNVWYADAIYPMYADTEVFSNLQRDMITCEAVQTLVTLVAQISETQYPVDRYLDWIPSLRASAATAATFAEWVNTSMKTAFDLSDMLLEPLLSGDPRMTQLAIQYQQYNGRTFNIIPEMPGSVIADCVQLTAEVFNHEYNLFGIARGDIIIGRVQSTHLWSPLAPPPDLVFDRDTPGVHIFGRDCRISFGMNGAAPMIRDETGLMVPFEGNWIFPLALWQMNTRYFNQQFDAWIKTGELRIRIEMGAYPYMLHYYDPRQYANAWNLTSAWLEEITPTSIPSVPFMVPISSDHDISSAPAVQYIISTEYNDRSLFCTNSSSPQTIAGPDKHIPVERYNILTNPDAPPTQIQLPEVVDLYNVVTRYAYETPPITAVVMGVP

SEQ ID NO: 22 - DNA, 3854 bp, reovirus
gctacacgttccacgacaatgtcatccatgatactgactcagtttggaccgttcattgagagcatttcaggtatcactgatcaatcgaatgacgtgtttgaagatgcagcaaaagcattctctatgtttactcgcagcgatgtctacaaggcgctggatgaaatacctttctctgatgatgcgatgcttccaatccctccaactatatatacgaaaccatctcacgattcatattattacattgatgctctaaaccgtgtgcgtcgcaaaacatatcagggccctgatgacgtgtacgtacctaattgttctattgttgaattgctggagccacatgagactctgacatcttatgggcggttgtccgaggccatcgagaatcgtgccaaggatggggacagccaagccagaatcgccacaacgtatggtagaatcgctgaatctcaagctcgacagattaaggctccattggagaagtttgtgttggcactattagtggccgaagcaggggggtctttatatgatccagttttgcagaagtatgatgagattccagatctatcgcataattgccctttatggtgttttagagagatctgtcgtcacatatctggtccattaccagatcgggcaccttatctttacttatctgcaggggtattctggttaatgtcaccacgaatgacgtctgcaatccctccgctactatccgatcttgttaatttagctattttgcaacaaactgcgggtttagatccatcattagtgaaattgggagtacagatatgccttcatgcagcagctagctcaagttattcatggtttatcttaaagactaagtctatttttcctcaaaacacgttgcacagtatgtatgaatctctagaagggggatactgtcctaatcttgaatggttagagcctagatcagactataagttcatgtacatgggagtcatgccattgtccgctaagtatgctaggtcggcgccgtccaatgataagaaagcgcgggaacttggcgagaaatatggactgagctcagtcgtcggtgagcttcgtaaacggacaaagacgtatgttaaacatgactttgcttcagtgaggtacattcgtgacgctatggcatgtactagcggtattttcttggtaagaacacccaccgaaacggtattgcaagaatatacgcagagtccggagattaaggttcccattccccagaaagactggacaggcccaataggtgaaatcagaattctaaaagatacaacaagttccatcgcgcgttacttatatagaacatggtacttggcagcggcgagaatggcggctcaaccacgtacgtgggatccattgtttcaagcgattatgagatctcaatacgtgacagctaggggtggatctggcgcagcactccgcgaatctttgtatgcaatcaatgtgtcgttacctgatttcaagggcttaccagtgaaggcagcaactaagatattccaggcggcacaattagcgaacttgccgttctcccacacatcagtggctatactagctgacacttcaatgggattgcgaaatcaggtgcagaggcggccacgatccattatgccattaaatgtgccccagcagcaggtttcggcgccccatacattgacagcggattacattaactaccacatgaatctatcacccacgtctggtagtgcggtcattgagaaggtgattcctttaggtgtatacgcttcgagccctcctaaccagtcgatcaacattgacatatctgcgtgtgacgctagtattacttgggatttctttctgtcagtgattatggcggctatacacgaaggtgtcgctagtagctccattggaaaaccatttatgggggttcctgcatccattgtaaatgatgagtctgtcgttggagtgagagctgctaggccgatatcgggaatgcagaacatgattcagcatctatcgaaactatataaacgtggattttcatatagagtaaacgattctttttctccaggtaacgattttactcatatgactaccactttcccgtcaggttcaacagccacctctactgagcatactgctaataatagtacgatgatggaaactttcctgacagtatggggacccgaacatactgacgaccctgacgtcttacgtttaatgaagtctttaactattcaaaggaattacgtatgtcaaggtgatgatggattaatgattatcgatgggactactgctggtaaggtgaacagtgaaactattcagaacgatctagaattaatctcaaaatatggtgaggaattcggatggaaatatgacatagcgtacgatgggactgccgaatacttaaagctatacttcatatttggctgtcgaattccaaatcttagtcgccatccaatcgtggggaaagaacgggcgaattcttcagcagaggagccatggccagcaattctagatcagattatgggtgtcttctttaatggtgttcatgatgggttacagtggcagcggtggatacgttattcatgggctctatgctgtgctttctcacgtcaaagaacaatgattggtgagagcgtgggttaccttcaatatcctatgtggtcttttgtctactggggattaccactggttaaagcgtttgggtcagacccatggatattttcttggtacatgcctactggagatctgggaatgtatagttggattagcttgatacgccctctgatgacaagatggatggtggctaatggttacgtaactgacagatgctcaaccgtattcgggaacgcagattatcgcaggtgtttcaatgaacttaaactatatcaaggttattatatggcacaattgcccaggaatcctaagaagtctggacgagcggcctctcgggaggtaagagaacaattcactcaggcattatccgactatctaatgcaaaatccagagctgaagtcacgtgtgctacgtggtcgtagtgagtgggagaaatatggagcggggataattcacaatcctccgtcattattcgatgtgccccataaatggtatcagggtgcgcaagaggcagcaatcgctacgagagaagagctggcagaaatggatgagacattaatgcgcgctcgaaggcacagctattcgagcttttcaaagttattagaggcgtatctgctcgtgaaatggcgaatgtgcgaggcccgcgaaccgtcggttgatttgcgattaccattatgtgcgggtattgacccattaaactcagatccttttctcaagatggtaagcgttggaccaatgctccagagtacgagaaagtactttgctcagacactattcatggcaaagacggtgtcgggtcttgacgttaacgcgattgatagcgcgttattacgactgcgaacattaggtgctgataagaaagcattaacggcgcagttattaatggtggggcttcaggagtcagaagcggacgcattggccgggaagataatgctacaggatgtgaatactgtgcaattagccagagtggttaacttagctgtgccagatacttggatgtcgttagactttgactctatgttcaaacaccacgtcaagctgcttcccaaagatggacgtcatctaaatactgatattcctcctcgaatgggatggttacgggccattttacgattcttaggtgccggaatggtaatgactgcgactggagttgctgtcgacatctatctggaggatatacatggcggtggtcggtcacttggacagagattcatgacttggatgcgacaggaaggacggtcagcgtgagtctaccatgggtcgtggtgcgtcaactcatc

SEQ ID NO: 23 - protein, 1267 aa, reovirus
MSSMILTQFGPFIESISGITDQSNDVFEDAAKAFSMFTRSDVYKALDEIPFSDDAMLPIPPTIYTKPSHDSYYYIDALNRVRRKTYQGPDDVYVPNCSIVELLEPHETLTSYGRLSEAIENRAKDGDSQARIATTYGRIAESQARQIKAPLEKFVLALLVAEAGGSLYDPVLQKYDEIPDLSHNCPLWCFREICRHISGPLPDRAPYLYLSAGVFWLMSPRMTSAIPPLLSDLVNLAILQQTAGLDPSLVKLGVQICLHAAASSSYSWFILKTKSIFPQNTLHSMYESLEGGYCPNLEWLEPRSDYKFMYMGVMPLSAKYARSAPSNDKKARELGEKYGLSSVVGELRKRTKTYVKHDFASVRYIRDAMACTSGIFLVRTPTETVLQEYTQSPEIKVPIPQKDWTGPIGEIRILKDTTSSIARYLYRTWYLAAARMAAQPRTWDPLFQAIMRSQYVTARGGSGAALRESLYAINVSLPDFKGLPVKAATKIFQAAQLANLPFSHTSVAILADTSMGLRNQVQRRPRSIMPLNVPQQQVSAPHTLTADYINYHMNLSPTSGSAVIEKVIPLGVYASSPPNQSINIDISACDASITWDFFLSVIMAAIHEGVASSSIGKPFMGVPASIVNDESVVGVRAARPISGMQNMIQHLSKLYKRGFSYRVNDSFSPGNDFTHMTTTFPSGSTATSTEHTANNSTMMETFLTVWGPEHTDDPDVLRLMKSLTIQRNYVCQGDDGLMIIDGTTAGKVNSETIQNDLELISKYGEEFGWKYDIAYDGTAEYLKLYFIFGCRIPNLSRHPIVGKERANSSAEEPWPAILDQIMGVFFNGVHDGLQWQRWIRYSWALCCAFSRQRTMIGESVGYLQYPMWSFVYWGLPLVKAFGSDPWIFSWYMPTGDLGMYSWISLIRPLMTRWMVANGYVTDRCSTVFGNADYRRCFNELKLYQGYYMAQLPRNPKKSGRAASREVREQFTQALSDYLMQNPELKSRVLRGRSEWEKYGAGIIHNPPSLFDVPHKWYQGAQEAAIATREELAEMDETLMRARRHSYSSFSKLLEAYLLVKWRMCEAREPSVDLRLPLCAGIDPLNSDPFLKMVSVGPMLQSTRKYFAQTLFMAKTVSGLDVNAIDSALLRLRTLGADKKALTAQLLMVGLQESEADALAGKIMLQDVNTVQLARVVNLAVPDTWMSLDFDSMFKHHVKLLPKDGRHLNTDIPPRMGWLRAILRFLGAGMVMTATGVAVDIYLEDIHGGGRSLGQRFMTWMRQEGRSA

SEQ ID NO: 24 - DNA, 1196 bp, reovirus
gctatttttgcctcttcccagacgttgtcgcaatggaggtgtgcttgcccaacggtcatcaggtcgtggacttgattaacaacgcttttgaaggtcgtgtatcaatctacagcgcgcaagagggatgggacaaaacaatctcagcacagccagatatgatggtatgtggtggcgccgtcgtttgcatgcattgtctaggtgttgttggatctctacaacgcaagctgaagcatttgcctcaccatagatgtaatcaacagatccgtcatcaggattacgtcgatgtacagttcgcagaccgtgttactgctcactggaagcggggtatgctgtccttcgttgcgcagatgcacgagatgatgaatgacgtgtcgccagatgacctggatcgtgtgcgtactgagggaggttcactagtggagctgaaccggcttcaggttgacccaaattcaatgtttagatcaatacactcaagttggacagatcctttgcaggtggtggacgaccttgacactaagctggatcagtactggacagccttaaacctgatgatcgactcatccgacttgatacccaactttatgatgagagacccatcacacgcgttcaatggtgtgaaactggagggagatgctcgtcaaacccaattctccaggacttttgattcgagatcgagtttggaatggggtgtgatggtttatgattactctgagctggagcatgatccatcgaagggccgtgcttacagaaaggaattggtgacgccagctcgagatttcggtcactttggattatcccattattctagggcgactaccccaatccttggaaagatgccggccgtattctcaggaatgttgactgggaactgtaaaatgtatccattcattaaaggaacggctaagctgaagacagtgcgcaagctagtggaggcagtcaatcatgcttggggtgtcgagaagattagatatgctcttgggccaggtggcatgacgggatggtacaataggactatgcaacaggcccccattgtgctaactcctgctgctctcacaatgttcccagataccatcaagtttggggatttgaattatccagtgatgattggcgatccgatgattcttggctaaacacccccatcttcacagcgccgggcttgaccaacctggtgtgacgtgggacaggcttcattcatc

SEQ ID NO: 25 - protein, 365 aa, reovirus
MEVCLPNGHQVVDLINNAFEGRVSIYSAQEGWDKTISAQPDMMVCGGAVVCMHCLGVVGSLQRKLKHLPHHRCNQQIRHQDYVDVQFADRVTAHWKRGMLSFVAQMHEMMNDVSPDDLDRVRTEGGSLVELNRLQVDPNSMFRSIHSSWTDPLQVVDDLDTKLDQYWTALNLMIDSSDLIPNFMMRDPSHAFNGVKLEGDARQTQFSRTFDSRSSLEWGVMVYDYSELEHDPSKGRAYRKELVTPARDFGHFGLSHYSRATTPILGKMPAVFSGMLTGNCKMYPFIKGTAKLKTVRKLVEAVNHAWGVEKIRYALGPGGMTGWYNRTMQQAPIVLTPAALTMFPDTIKFGDLNYPVMIGDPMILG

SEQ ID NO: 26 - DNA, 2203 bp, reovirus
gctaatctgctgaccgttactctgcaaagatggggaacgcttcctctatcgttcagacgatcaacgtcactggagatggcaatgtatttaaaccatcagctgaaacttcatctaccgctgtaccatcgttaagcttatcacctggaatgctgaatcccggaggggtaccatggattgctgttggagatgagacatctgtgacttcaccaggcgcattacgtcgaatgacgtcaaaggacatcccggaaacggcaataatcaacacagacaattcatcaggcgccgtgccaagcgaatcagcgcttgtgccctacatcgatgagccgctggtagtggttacagagcatgctattaccaacttcaccaaagctgagatggcacttgaattcaatcgtgagttccttgacaagatgcgtgtgctgtcagtgtcaccaaaatattcggatcttctgacctatgttgactgctacgtcggtgtgtctgctcgtcaggctttaaacaattttcagaaacaagtgcctgtgattacacctactaggcagacgatgtatgtcgactcgatacaagcggccttgaaagctttagaaaagtgggagattgatctgagagtggctcaaacgttgctgcctacgaacgttccgattggagaagtctcttgtccaatgcagtcggtagtgaaactgctggatgatcagctgccagatgacacgctgatacggaggtatcccaaggaagccgccgtcgctttggctaaacgaaacgggggaatacaatggatggacgtatcagaaggcaccgtgatgaacgaggctgtcaacgctgttgcagctagtgcactggcaccttcagcatcagccccacccttagaagagaagtcaaagttaaccgaacaagcgatggatctcgtgaccgcggctgagcctgagataattgcctcactcgcgccagttcccgcacccgtgtttgccataccacctaaaccagcagattataatgtgcgtactctgaggatcgacgaggccacttggctgcgaatgattccaaaatcaatgaacacaccttttcaaatccaggtgactgataacacaggaactaattggcatctcaatttgaggggggggactcgtgtagtgaatctggaccaaatcgctccgatgcggtttgtattagatttagggggaaagagttataaagagacgagctgggatccaaacggcaagaaggtcggattcatcgtttttcaatcgaagataccattcgaactttggactgctgcttcacagatcggtcaagccacggtggttaactatgtccaactatacgctgaagacagctcatttaccgcgcagtctatcattgctactacctctttggcttataactatgagcctgagcagttgaataagactgaccctgagatgaattattatcttttggcgacctttatagactcagccgctataacgccaacgaatatgacacagcctgatgtttgggatgccttgctgacgatgtccccactatcagctggcgaggtgacagtgaagggtgcggtagtgagtgaagtagtccctgcagacttgataggtagctacactccagaatccctaaacgcctcacttccgaatgatgctgctagatgcatgatcgatagagcttcgaagatagccgaagcaatcaagattgatgatgatgctggaccagatgaatattccccaaactctgtaccaattcaaggtcagcttgctatctcgcaactcgaaactggatatggtgtgcgaatattcaaccctaaagggatcctttctaaaattgcatctagggcaatgcaggctttcattggtgacccgagcacaatcatcacgcaggcggcgccagtgttatcagacaagaataattggattgcattggcacagggagtgaaaactagtctgcgtactaaaagtctatcagcgggagtgaagactgcagtgagtaagctgagctcatctgagtctatccagaattggactcaaggattcttggataaagtgtcagcgcattttccagcaccaaagcccgattgtccgactagcggagatagtggtgaatcgtctaatcgccgagtgaagcgcgactcatacgcaggagtggtcaaacgtgggtacacacgttaggccgctcgccctggtgacgcggggttaagggatgcaggcaaatcatc

SEQ ID NO: 27 - protein, 708 aa, reovirus
MGNASSIVQTINVTGDGNVFKPSAETSSTAVPSLSLSPGMLNPGGVPWIAVGDETSVTSPGALRRMTSKDIPETAIINTDNSSGAVPSESALVPYIDEPLVVVTEHAITNFTKAEMALEFNREFLDKMRVLSVSPKYSDLLTYVDCYVGVSARQALNNFQKQVPVITPTRQTMYVDSIQAALKALEKWEIDLRVAQTLLPTNVPIGEVSCPMQSVVKLLDDQLPDDTLIRRYPKEAAVALAKRNGGIQWMDVSEGTVMNEAVNAVAASALAPSASAPPLEEKSKLTEQAMDLVTAAEPEIIASLAPVPAPVFAIPPKPADYNVRTLRIDEATWLRMIPKSMNTPFQIQVTDNTGTNWHLNLRGGTRVVNLDQIAPMRFVLDLGGKSYKETSWDPNGKKVGFIVFQSKIPFELWTAASQIGQATVVNYVQLYAEDSSFTAQSIIATTSLAYNYEPEQLNKTDPEMNYYLLATFIDSAAITPTNMTQPDVWDALLTMSPLSAGEVTVKGAVVSEVVPADLIGSYTPESLNASLPNDAARCMIDRASKIAEAIKIDDDAGPDEYSPNSVPIQGQLAISQLETGYGVRIFNPKGILSKIASRAMQAFIGDPSTIITQAAPVLSDKNNWIALAQGVKTSLRTKSLSAGVKTAVSKLSSSESIQNWTQGFLDKVSAHFPAPKPDCPTSGDSGESSNRRVKRDSYAGVVKRGYTR

SEQ ID NO: 28 - DNA, 2304 bp, reovirus
gctattcgcggtcatggcttacatcgcagttcctgcggtggtggattcacgttcgagtgaggctattggactgctagaatcgtttggagtagacgctggggctgacgcgaatgacgtttcatatcaagatcatgactatgtgttggatcagttacagtacatgttagatggatatgaggctggtgacgttatcgatgcactcgtccacaagaattggttacatcactctgtctattgcttgttgccacccaaaagtcaactattagagtattggaaaagtaatccttcagcgataccggacaacgttgatcgtcggcttcgtaaacgactaatgctaaagaaagatctcaggaaagatgatgaatacaatcagctagcgcgtgctttcaagatatcggatgtctacgcacctctcatctcatccacgacgtcaccgatgacaatgatacagaacttgaatcgaggcgagatcgtgtacaccacgacggacagggtaataggggctagaatcttgttatatgctcctagaaagtactatgcgtcaactctgtcatttactatgactaagtgcatcattccgtttggtaaagaggtgggtcgtgttcctcactctcgatttaatgttggcacatttccgtcaattgctaccccgaaatgttttgtcatgagtggggttgatattgagtccatcccaaatgaatttatcaagttgttttaccagcgcgtcaagagtgttcacgctaacatactaaatgacatatctcctcagatcgtctctgacatgataaacagaaagcgtctgcgcgttcatactccatcagatcgtcgagccgcgcagttgatgcatttgccttaccatgttaaacgaggagcgtctcacgtcgacgtttacaaggtggatgttgtagacatgttgttcgaggtagtggatgtggccgatgggttgcgcaacgtatctaggaaactaactatgcataccgttcctgtatgtattcttgaaatgttgggtattgagattgcggactattgcattcgtcaagaggatggaatgctcacagattggttcctacttttaaccatgctatctgatggcttgactgatagaaggacgcattgtcaatacttgatgaatccgtcaagtgtgcctcctgatgtgatacttaacatctcaattactggatttataaatagacatacaatcgatgtcatgcctgacatatatgacttcgttaaacccattggcgctgtgctgcctaagggatcatttaaatcaacaattatgagagttcttgattcaatatcaatattaggaatccaaatcatgccgcgcgcgcatgtagttgactcagatgaggtgggcgagcaaatggagcctacgtttgagcaggcggttatggagatatacaaagggattgctggcgttgactcgctggatgatctcatcaagtgggtgttgaactcggatctcattccgcatgatgacaggcttggtcaattatttcaagcgtttttgcctctcgcaaaggacttattagctccaatggccagaaagttttatgataactcaatgagtgagggtagattgctaacattcgctcatgccgacagtgagttgctgaacgcaaattattttggtcatttattgcgactaaaaataccatatattacagaggttaatctgatgattcgcaagaatcgtgagggtggagagctatttcagcttgtgttatcttatctatataaaatgtatgctactagcgcgcagcctaaatggtttggatcattattgcgattgttaatatgtccctggttacatatggagaaattaataggagaagcagacccggcatctacgtcggctgaaattgggtggcatatccctcgtgaacagctgatgcaagatggatggtgtggatgtgaagacggattcattccctatgttagcatacgtgcgccaagactggttatagaggagttgatggagaagaactggggccaatatcatgcccaagttattgtcactgatcagcttgtcgtaggcgaaccgcggagggtatctgctaaggctgtgatcaagggtaaccacttaccagttaagttagtttcacgatttgcatgtttcacattgacggcgaagtatgagatgaggctttcgtgcggccatagcactggacgtggagctgcatacagtgcgagactagctttccgatctgacttggcgtgatccgtgacatgcgtagtgtgacacctgctcctaggtcaatgggggtagggggcgggctaagactacgtacgcgcttcatc

SEQ ID NO: 29 - protein, 736 aa, reovirus
MAYIAVPAVVDSRSSEAIGLLESFGVDAGADANDVSYQDHDYVLDQLQYMLDGYEAGDVIDALVHKNWLHHSVYCLLPPKSQLLEYWKSNPSAIPDNVDRRLRKRLMLKKDLRKDDEYNQLARAFKISDVYAPLISSTTSPMTMIQNLNRGEIVYTTTDRVIGARILLYAPRKYYASTLSFTMTKCIIPFGKEVGRVPHSRFNVGTFPSIATPKCFVMSGVDIESIPNEFIKLFYQRVKSVHANILNDISPQIVSDMINRKRLRVHTPSDRRAAQLMHLPYHVKRGASHVDVYKVDVVDMLFEVVDVADGLRNVSRKLTMHTVPVCILEMLGIEIADYCIRQEDGMLTDWFLLLTMLSDGLTDRRTHCQYLMNPSSVPPDVILNISITGFINRHTIDVMPDIYDFVKPIGAVLPKGSFKSTIMRVLDSISILGIQIMPRAHVVDSDEVGEQMEPTFEQAVMEIYKGIAGVDSLDDLIKWVLNSDLIPHDDRLGQLFQAFLPLAKDLLAPMARKFYDNSMSEGRLLTFAHADSELLNANYFGHLLRLKIPYITEVNLMIRKNREGGELFQLVLSYLYKMYATSAQPKWFGSLLRLLICPWLHMEKLIGEADPASTSAEIGWHIPREQLMQDGWCGCEDGFIPYVSIRAPRLVIEELMEKNWGQYHAQVIVTDQLVVGEPRRVSAKAVIKGNHLPVKLVSRFACFTLTAKYEMRLSCGHSTGRGAAYSARLAFRSDLA

SEQ ID NO: 30 - protein, 418 aa, reovirus; identical to SEQ ID NO: 12

(19) United States
(12) Patent Application Publication
(10) Pub. No.: US 2022/0105143 A1
(43) Pub. Date: Apr. 7, 2022

(54) T CELL REPERTOIRE DYNAMICS AND ONCOLYTIC VIRAL THERAPY

(71) Applicant: Oncolytics Biotech Inc., Calgary (CA)

(72) Inventor: Grey Wilkinson, Calgary (CA)

(21) Appl. No.: 17/427,446

(22) PCT Filed: Feb. 21, 2020
(86) PCT No.: PCT/IB2020/051493

§ 371 Date: Jul. 30, 2021

Related U.S. Application Data
(60) Provisional application No. 62/809,190, filed on Feb. 22, 2019.

(51) Int. Cl. A61K 35/765; A61K 39/395; A61P 35/00; A61K 31/4196

(52) U.S. Cl. CPC A61K 35/765; A61K 31/4196; A61P 35/00; A61K 39/3955

(57) ABSTRACT

Provided herein are methods of treating cancer in a subject. The methods include administering to the subject one or more doses of an oncolytic virus (e.g., in an initial round of treatment); selecting a subject with a T-cell population exhibiting high peripheral clonality; and administering to the subject with a T-cell population exhibiting high peripheral clonality a one or more subsequent doses of the oncolytic virus (e.g., in a second round of treatment).

### CROSS REFERENCE TO RELATED APPLICATION

This application claims priority to U.S. Provisional Application No. 62/809,190, filed Feb. 22, 2019, which is incorporated by reference herein in its entirety.

### BACKGROUND

Cancer is one of the leading causes of death. Although it has long been the focus of medical research, main cancer therapies remain surgery, radiation therapy and chemotherapy. Each one of these therapies is subject to limitations including, for example, the differing effects of the same therapy to subjects with similar types of cancer.

### BRIEF SUMMARY

Provided herein are methods of treating cancer in a subject, and, more particularly, a method of treating cancer in a subject selected for likely treatment success by determining whether the subject demonstrates a T-cell population exhibiting high peripheral clonality after a first round of treatment. The methods include administering to a subject with cancer one or more doses of an oncolytic virus, such as a reovirus; selecting a subject with a T-cell population exhibiting high peripheral clonality (e.g., greater than 0.06) after treatment with one or more doses of the oncolytic virus; and administering to the subject with a T-cell population exhibiting high peripheral clonality one or more subsequent doses of the oncolytic virus. Optionally, the oncolytic virus is administered in combination with one or more additional agents.

The details of one or more embodiments are set forth in the accompanying description below. Other features, objects, and advantages will be apparent from the description and from the claims.

### BRIEF DESCRIPTION OF THE DRAWINGS

FIG. 1 is a graph showing a trend towards decreased peripheral clonality over treatment using the paired Wilcox rank sum test.

FIG. 2 is a graph showing a trend towards increased peripheral diversity over treatment using the paired wilcox rank sum test.

FIG. 3 is a graph showing higher peripheral clonality and lower diversity at C1D1 (Day 1 of treatment cycle 1) and C2D1 (C2D1, Day 1 of treatment cycle 2) as correlated with progression free survival.

FIG. 4 is a graph showing higher peripheral clonality and lower diversity at C1D1 and C2D1 as correlated with overall survival.

FIG. 5 is a graph showing peripheral T cell fraction over time.

FIG. 6 is a graph showing the Morisita index relative to C1D1.

FIG. 7 is a graph showing peripheral clonal expansion at C2D1.

FIG. 8 is a graph showing the majority of peripherally expanded clones identified at C2D1 are from new clones.

FIGS. 9A and 9B are graphs showing higher peripheral clonality is correlated with greater changes in CelTIL score in breast cancer patients treated with reovirus and letrozole (9A) and breast cancer patients treated with reovirus and checkpoint inhibitor atezolizumab (9B).

### DETAILED DESCRIPTION OF THE INVENTION

Provided herein is a method of treating cancer in a subject by selecting a subject having one or more markers that indicate the subject will be responsive to the treatment by showing, for example, an enhanced overall survival time and/or enhanced progression free survival time. The method includes administering to a subject one or more doses of an oncolytic virus (i.e., a first round of treatment with the oncolytic virus), selecting a subject with a T-cell population exhibiting high peripheral clonality after one or more doses of the oncolytic virus, and administering to the selected subject a subsequent one or more doses of the oncolytic virus (i.e., a second round of treatment with the oncolytic virus). Optionally, the subject also has a T cell population with low diversity. Selection of the subject with a T cell population with high peripheral clonality and low diversity results in selection of subject that, upon subsequent treatment with the oncolytic virus (i.e., a second round of treatment) shows longer progression free survival and/or overall survival as compared to subjects without selection or as compared to subjects lacking a T cell population with high peripheral clonality and low diversity after one or more doses of the oncolytic virus.

As used herein clonality refers to the quantitation of the extent of mono- or oligoclonal expansion by measuring the shape of the clone frequency distribution. The values of clonality range from 0 to 1, where values approaching 1 indicate a nearly monoclonal population. Generally, as used herein, high clonality refers to values of about 0.06 or higher. The term diversity refers to the number of unique rearrangements. Generally, as used herein, low diversity refers to the T cell population diversity of less than about 1800 rearrangements.

Clonality and diversity can be calculated in various ways. By way of example the following equations can be used:

$$Diversity = H = - {\sum }_{i = 1}^{N} {p}_{i} {log}_{2} ( {p}_{i} )$$

$$Clonality = 1 - \frac{H}{{log}_{2} ( N )}$$

pᵢ is the proportional abundance of clone i, and N is the total number of unique receptor gene rearrangements.

Alternatively, clonality can also be calculated with Simpson clonality:

Simpson Clonality=\sqrt{Σpᵢ²}

pᵢ is the proportional abundance of clone i.

As used herein, the term cancer refers to all types of cancer, proliferative disorders, neoplasia, or malignant tumors found in mammals, including lymphomas, leukemias, blastomas, germ cell tumors, carcinomas, and sarcomas. Exemplary cancers include cancer of the brain, breast, cervix, colon, head and neck, liver, kidney, lung, non-small cell lung, melanoma, mesothelioma, ovary, sarcoma, stomach, uterus, and medulloblastoma. Optionally, the cancer is a neoplasm. Optionally, the cancer is head and neck cancer. Optionally, the cancer is lung cancer, liver cancer, lymphoma, pancreatic cancer, melanoma, kidney cancer or ovarian cancer. Optionally, the cancer is an adenocarcinoma. Optionally, the cancer is a pancreatic adenocarcinoma.

Optionally, the cancer is metastatic. As used herein, the terms metastasis, metastatic, and metastatic cancer can be used interchangeably and refer to the spread of a proliferative disease or disorder, e.g., the spread of cancer from one organ to another non-adjacent organ or body part. Cancer occurs at an originating site, e.g., pancreas, which site is referred to as a primary tumor, e.g., primary pancreatic cancer. Some cancer cells in the primary tumor or originating site acquire the ability to penetrate and infiltrate surrounding normal tissue in the local area and/or the ability to penetrate the walls of the lymphatic system or vascular system to circulate to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if pancreatic cancer metastasizes to the lung, the secondary tumor at the site of the lung consists of abnormal pancreatic cells and not abnormal lung cells. The secondary tumor in the lung is referred to a metastatic pancreatic cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but no secondary tumor. For example, metastatic pancreatic cancer refers to a disease in a subject with or with a history of a primary pancreatic tumor and with one or more secondary tumors at a second location or multiple locations, e.g., in the lung.

Oncolytic viruses that are used in the provided methods and kits include, but are not limited to, oncolytic viruses that are members in the family of reoviridae, myoviridae, siphoviridae, podpviridae, teciviridae, corticoviridae, plasmaviridae, lipothrixviridae, fuselloviridae, poxyiridae, iridoviridae, phycodnaviridae, baculoviridae, herpesviridae, adnoviridae, papovaviridae, polydnaviridae, inoviridae, microviridae, geminiviridae, circoviridae, parvoviridae, hepadnaviridae, retroviridae, cyctoviridae, birnaviridae, paramyxoviridae, rhabdoviridae, Filoviridae, orthomyxoviridae, bunyaviridae, arenaviridae, leviviridae, picornaviridae, sequiviridae, comoviridae, potyviridae, caliciviridae, astroviridae, nodaviridae, tetraviridae, tombusviridae, coronaviridae, glaviviridae, togaviridae, and barnaviridae. Immunoprotected viruses and reassortant or recombinant viruses of these and other oncolytic viruses are also encompassed by the provided methods. Thus, the oncolytic virus used in the provided methods is, for example, selected from the group consisting of a reovirus, a Newcastle disease virus (NDV), a vesicular stomatitis virus (VSV), an adenovirus, a vaccinia virus, a parapox orf virus, a Sindbis virus, and a herpes simplex virus. Furthermore, a combination of at least two oncolytic viruses can also be employed to practice the provided methods. A few oncolytic viruses are discussed below, and a person of ordinary skill in the art can practice the present methods using other oncolytic viruses as well according to the disclosure herein and knowledge available in the art.

When a virus enters a cell, double-stranded RNA Kinase (PKR) is activated, blocking protein synthesis, and the virus cannot replicate in this cell. Some viruses have developed a system to inhibit PKR and to facilitate viral protein synthesis as well as viral replication. For example, adenovirus makes a large amount of a small RNA, VA1 RNA. VA1 RNA has extensive secondary structures and binds to PKR in competition with the double-stranded RNA (dsRNA) that normally activates PKR. Since a minimum length of dsRNA is required to activate PKR, VA1 RNA does not activate PKR. Instead, it sequesters PKR by virtue of its large amount. Consequently, protein synthesis is not blocked, and adenovirus can replicate in the cell. Accordingly, if the PKR inhibitors in adenovirus, vaccinia virus, herpes simplex virus, or parapoxvirus orf virus are mutated so as not to block PKR function anymore, the resulting viruses do not infect normal cells due to protein synthesis inhibition by PKR, but they replicate in cancer cells lacking PKR activities. Optionally, the oncolytic virus is an adenovirus mutated in the VA1 region, a vaccinia virus mutated in the K3L and/or E3L region, a vaccinia virus mutated in the thymidine kinase (TK) gene, a vaccinia virus mutated in the vaccinia growth factor (VGF) gene, a herpes virus mutated in the y134.5 gene, a parapoxvirus orf virus mutated in the OV20.0L gene, or an influenza virus mutated in the NS-1 gene.

Vaccinia viruses mutated in the viral thymidine kinase (TK) gene are unable to make nucleotides needed for DNA replication. In normal cells, the cellular TK levels are usually very low and the virus is unable to replicate. In tumors, loss of the tumor suppressor Rb or an increase in cyclin activity leads to E2F pathway activation and high levels of TK expression. Thus, cancer cells have high TK levels and the mutated vaccinia virus can replicate and spread.

The vaccinia growth factor (VGF) gene is a homolog of mammalian epidermal growth factor (EGF) and can bind and activate the EGF Receptor (EGFR). Vaccinia viruses mutated in the VGF gene are growth restricted to cells with activated EGF pathways, which is commonly mutated in cancers.

The viruses can be modified or mutated according to the known structure-function relationship of the viral PKR inhibitors. For example, since the amino terminal region of E3 protein interacts with the carboxy-terminal region domain of PKR, deletion or point mutation of the carboxy-terminal region domain prevents anti-PKR function (Chang et al., PNAS 89:4825-4829 (1992); Chang et al., Virology 194:537-547 (1993); Chang et al., J. Virol. 69:6605-6608 (1995); Sharp et al., Virol. 250:301-315 (1998); and Romano et al., Mol. and Cell. Bio. 18:7304-7316 (1998)). The K3L gene of vaccinia virus encodes pK3, a pseudosubstrate of PKR. Truncations or point mutations within the C-terminal portion of K3L protein that is homologous to residues 79 to 83 in eIF-2 abolish PKR inhibitory activity (Kawagishi-Kobayashi et al., Mol. Cell. Biology 17:4146-4158 (1997)).

Another example is the Delta24 virus, which is a mutant adenovirus carrying a 24 base pair deletion in the E1A region. (Fueyo et al., Oncogene 19(1):2-12 (2000)). This region is responsible for binding to the cellular tumor suppressor Rb and inhibiting Rb function, thereby allowing the cellular proliferative machinery, and hence virus replication, to proceed in an uncontrolled fashion. Delta24 has a deletion in the Rb binding region and does not bind to Rb. Therefore, replication of the mutant virus is inhibited by Rb in a normal cell. However, if Rb is inactivated and the cell becomes neoplastic, Delta24 is no longer inhibited. Instead, the mutant virus replicates efficiently and lyses the Rb-deficient cell.

In addition, vesicular stomatitis virus (VSV) selectively kills neoplastic cells. A herpes simplex virus 1 (HSV-1) mutant defective in ribonucleotide reductase expression, hrR3, replicates in colon carcinoma cells but not normal liver cells (Yoon et al., FASEB J. 14:301-311(2000)). Newcastle disease virus (NDV) replicates preferentially in malignant cells, and the most commonly used strain is 73-T (Reichard et al., J. Surgical Research 52:448-453 (1992); Zorn et al., Cancer Biotherapy 9(3):22-235 (1994); Bar-Eli et al., J. Cancer Res. Clin. Oncol. 122: 409-415 (1996)). Vaccinia virus propagates in several malignant tumor cell lines. Encephalitis virus has an oncolytic effect in a mouse sarcoma tumor, but attenuation may be required to reduce its infectivity in normal cells. Tumor regression has been described in tumor patients infected with herpes zoster, hepatitis virus, influenza, varicella, and measles virus (for a review, see Nemunaitis, J. Invest. New Drugs 17:375-386 (1999)).

Optionally, the oncolytic virus is a modified, non-reovirus virus comprising a reovirus sigma 1 protein, wherein the reovirus sigma 1 protein replaces the native attachment protein of the non-reovirus virus and wherein the modified virus does not comprise any portion of the native attachment protein of the non-reovirus virus. In the modified, non-reovirus virus, the reovirus sigma 1 protein attaches to carrier cells that protect the virus from neutralizing antibodies during in vivo delivery to a tumor, for example, during systemic delivery. The non-reovirus virus can be, but is not limited to, an adenovirus, a vaccinia virus, a herpes simplex virus, a Sindbis virus, or a parapox virus. Optionally, the full-length sequence of the native attachment protein of the non-reovirus virus is replaced with a reovirus sigma 1 protein. Replacement of the native attachment protein of the virus with a reovirus sigma 1 protein allows the virus to attach to carrier cells which protect the virus from neutralizing antibodies during in vivo delivery. The reovirus sigma-1 protein is described in, for example, WO 2008/11004, which is incorporated by reference herein in its entirety.

Optionally, the oncolytic virus is a reovirus. Reovirus refers to any virus classified in the reovirus genus, whether naturally occurring, modified, or recombinant. Reoviruses are viruses with a double-stranded, segmented RNA genome. The virions measure 60-80 nm in diameter and possess two concentric capsid shells, each of which is icosahedral. The genome consists of double-stranded RNA in 10-12 discrete segments with a total genome size of 16-27 kbp. The individual RNA segments vary in size. Three distinct but related types of reoviruses have been recovered from many species. Thus, the reovirus can be a mammalian reovirus or a human reovirus. All three types share a common complement-fixing antigen.

Human reovirus includes three serotypes: type 1 (strain Lang or T1L), type 2 (strain Jones, T2J), and type 3 (strain Dearing or strain Abney, T3D). The three serotypes are easily identifiable on the basis of neutralization and hemagglutinin-inhibition assays. A reovirus according to this disclosure can be a type 3 mammalian orthoreovirus. Type 3 mammalian orthoreoviruses include, without limitation, Dearing and Abney strains (T3D or T3A, respectively). See, for example, ATCC Accession Nos. VR-232 and VR-824. See, for example, U.S. Pat. Nos. 6,110,461; 6,136,307; 6,261,555; 6,344,195; 6,576,234; and 6,811,775, which are incorporated by reference herein in their entireties.

Optionally, the provided methods include the use of reoviruses with mutations. For example, mutant reoviruses as described herein can contain a mutation that reduces or essentially eliminates expression of a sigma3 polypeptide or that results in the absence of a functional sigma3 polypeptide as described in U.S. Publication No. 2008/0292594, which is incorporated by reference herein in its entirety. A mutation that eliminates expression of a sigma3 polypeptide or that results in the absence of a functional sigma3 polypeptide can be in the nucleic acid encoding the sigma3 polypeptide (i.e., the S4 gene) or in a nucleic acid that encodes a polypeptide that regulates the expression or function of the sigma3 polypeptide.

As used herein, a mutation that reduces the expression of a sigma3 polypeptide refers to a mutation that results in a decrease in the amount of sigma3 polypeptide, compared to a reovirus expressing wild type levels of sigma3 polypeptide, of at least 30% (e.g., at least 40%, 50%, 60%, 70%, 80%, 90%, or 95%). As used herein, a mutation that essentially eliminates expression of a sigma3 polypeptide refers to a mutation that results in a decrease in the amount of sigma3 polypeptides, relative to the amount of sigma3 polypeptides produced by a wild type reovirus, of at least 95% (e.g., 96%, 97%, 98%, 99%, or 100%). As used herein, a mutation that results in a decrease in or absence of a functional sigma3 polypeptide refers to a mutation that allows expression of the sigma3 polypeptide but that results in a sigma3 polypeptide that is not able to assemble or incorporate into the viral capsid. It would be understood that it may be desirable or necessary for sigma3 polypeptides to retain other functionalities (e.g., the ability to bind RNA) in order for the mutant reovirus to retain the ability to propagate.

A mutation in a sigma3 polypeptide as described herein can result in a sigma3 polypeptide that is incorporated into the capsid at levels that are reduced relative to a sigma3 polypeptide that does not contain the mutation (e.g., a wild type sigma3 polypeptide). A mutation in a sigma3 polypeptide as described herein also can result in a sigma3 polypeptide that cannot be incorporated into a viral capsid. Without being bound by any particular mechanism, a sigma3 polypeptide may have reduced function or lack function due, for example, to an inability of the sigma3 polypeptide and the mu1 polypeptide to bind appropriately, or due to a conformational change that reduces or prohibits incorporation of the sigma3 polypeptide into the capsid.

In addition to a mutation that abolishes or reduces expression of the sigma3 polypeptide or that results in a non-functional or reduced-function sigma3 polypeptide, a mutant reovirus as described herein may contain one or more further mutations (e.g., a second, third, or fourth mutation) in one of the other reovirus capsid polypeptides (e.g., mu1, lambda2, and/or sigma1). Reoviruses containing a mutation affecting the sigma3 polypeptide and, optionally, a further mutation in any or all of the other outer capsid proteins can be screened for the ability of such mutant reoviruses to infect and cause lysis of cells. For example, neoplastic cells that are resistant to lysis by wild type reovirus can be used to screen for effective mutant reoviruses described herein.

For example, a further mutation can reduce or essentially eliminate expression of a mu1 polypeptide or result in the absence of a functional mu1 polypeptide. The mu1 polypeptide, which is encoded by the M2 gene, is likely involved in cell penetration and may play a role in transcriptase activation. Each virion contains about 600 copies of mu1 polypeptide, which are present in the form of 1:1 complexes with sigma3 polypeptides. The mu1 polypeptide is myristolated on its N-terminus, and then the myristolated N-terminal 42 residues are cleaved off, resulting in a C-terminal fragment (mu1C). Additionally or alternatively, a further mutation can reduce or essentially eliminate expression of a lambda2 polypeptide or result in the absence of a functional lambda2 polypeptide, and/or a further mutation can reduce or essentially eliminate expression of a sigma1 polypeptide or result in the absence of a functional sigma1 polypeptide. The lambda2 polypeptide is encoded by the L2 gene, is involved in particle assembly, and exhibits guanylyltransferase and methyltransferase activity. The sigma1 polypeptide is encoded by the Si gene, is involved in cell-attachment and serves as the viral hemagglutinin.

Optionally, the reovirus comprises a lambda-3 polypeptide having one or more amino acid modifications, a sigma-3 polypeptide having one or more amino acid modifications, a mu-1 polypeptide having one or more amino acid modifications, a mu-2 polypeptide having one or more amino acid modifications, or any combination thereof. For example, the reovirus has a lambda-3 polypeptide having one or more amino acid modifications; a sigma-3 polypeptide having one or more amino acid modifications; a mu-1 polypeptide having one or more amino acid modifications; and/or a mu-2 polypeptide having one or more amino acid modifications, as described in U.S. Ser. No. 12/046,095, which is incorporated by reference herein in its entirety. By way of example, the one or more amino acid modifications in the lambda-3 polypeptide are a Val at residue 214, an Ala at residue 267, a Thr at residue 557, a Lys at residue 755, a Met at residue 756, a Pro at residue 926, a Pro at residue 963, a Leu at residue 979, an Arg at residue 1045, a Val at residue 1071, or any combination thereof, numbered relative to GenBank Accession No. M24734.1 (SEQ ID NO:23). It is noted that, when the amino acid sequence is a Val at residue 214 or a Val at residue 1071, the amino acid sequence further includes at least one additional change in the amino acid sequence. Optionally, the lambda-3 polypeptide includes the sequence shown in SEQ ID NO:19. Further by way of example, the one or more amino acid modifications in the sigma-3 polypeptide are a Leu at residue 14, a Lys at residue 198, or any combination thereof, numbered relative to GenBank Accession No. K02739 (SEQ ID NO:25). It is noted that, when the amino acid sequence is a Leu at residue 14, the amino acid sequence further includes at least one additional change in the amino acid sequence. Optionally, the sigma-3 polypeptide includes the sequence shown in SEQ ID NO:15. Further by way of example, the one or more amino acid modifications in the mu-1 polypeptide is an Asp at residue 73 numbered relative to GenBank Accession No. M20161.1 (SEQ ID NO:27). Optionally, the mu-1 polypeptide includes the sequence shown in SEQ ID NO:17. Also by way of example, the amino acid modification mu-2 polypeptide is a Ser at residue 528 numbered relative to GenBank Accession No. AF461684.1 (SEQ ID NO:29). Optionally, the mu-1 polypeptide includes the sequence shown in SEQ ID NO:17. A reovirus as described herein having one or more modifications can further include a reovirus sigma-2 polypeptide. Such a sigma-2 polypeptide has a Cys at one or more of position 70, 127, 195, 241, 255, 294, 296, or 340, numbered relative to GenBank Accession No. NP_694684.1 (SEQ ID NO:30). Optionally, the sigma-2 polypeptide includes the sequence shown in SEQ ID NO:12.

Optionally, the reovirus comprises a L1 genome segment comprising one or more nucleic acid modifications, an S4 genome segment comprising one or more nucleic acid modifications, an M1 genome segment comprising one or more nucleic acid modifications, an M2 genome segment comprising one or more nucleic acid modifications, or any combination thereof. Optionally, the reovirus has a L1 genome segment having one or more nucleic acid modifications; a S4 genome segment having one or more nucleic acid modifications; a M1 genome segment having one or more nucleic acid modifications; and/or a M2 genome segment having one or more nucleic acid modifications, as described in WO 2008/110004, which is incorporated by reference herein in its entirety. By way of example, the one or more nucleic acid modifications in the L1 genome segment are a T at position 660, a G at position 817, an A at position 1687, a G at position 2283, an ATG at positions 2284-2286, a C at position 2794, a C at position 2905, a C at position 2953, an A at position 3153, or a G at position 3231, numbered relative to GenBank Accession No. M24734.1 (SEQ ID NO:22). Optionally, the L1 genome segment includes the sequence shown in SEQ ID NO:8. Further by way of example, the one or more nucleic acid modifications in the S4 genome segment is an A at position 74 and an A at position 624, numbered relative to GenBank Accession No. K02739 (SEQ ID NO:24). Optionally, the S4 genome segment includes the sequence shown in SEQ ID NO:4. Further by way of example, the nucleic acid modification in the M2 genome segment can be a C at position 248, numbered relative to GenBank Accession No. M20161.1 (SEQ ID NO:26). The M2 genome segment, for example, includes the sequence shown in SEQ ID NO:6. Also by way of example, the nucleic acid modification in the M1 genome segment is a T at position 1595, numbered relative to GenBank Accession No. AF461684.1 (SEQ ID NO:28). Optionally, the M1 genome segment includes the sequence shown in SEQ ID NO:5. A reovirus as described herein can include any modification or combination of modifications disclosed herein. Optionally, a reovirus as described herein includes genomic segments having the sequences shown in SEQ ID NOs:1-10 or the polypeptides shown in SEQ ID NOs:11, 12, and 16-21, and either or both SEQ ID NO:13 or 14. Optionally, a reovirus as disclosed herein is identified as IDAC Accession No. 190907-01, which was deposited with the International Depositary of Canada (IDAC, National Microbiology Laboratory, Public Health Agency of Canada, 1015 Arlington St., Winnipeg, Manitoba Canada R3E 3R2 on Sep. 19, 2007.

Sindbis virus (SIN) can be used in the methods described herein. Sindbis virus is a member of the alphavirus genus of the togaviridae family. The Sindbis virus genome is a single-stranded RNA of 11703 nucleotides, capped at the 5′ terminus and poly-adenylated at the 3′ terminus. The genome consists of a 49S untranslated region (UT), nonstructural proteins nsP1, nsP2, nsP3, and nsP4 followed by a promoter. The promoter is followed by a 26S UT, structural proteins C, E3, E2, 6K, and E1 and finally a 3′ UT and a poly-adenylated terminus. The genomic 49S RNA is of plus sense, is infectious, and serves as mRNA in the infected cell.

Sindbis vectors systemically and specifically infect/detect and kill metastasized tumors in vivo, leading to significant suppression of tumor growth and enhanced survival (Hurtado et al., Rejuvenation Res. 9(1):36-44 (2006)). Sindbis virus infects mammalian cells using the Mr 67,000 laminin receptor, which is elevated in tumor versus normal cells. Tumor overexpression of the laminin receptor may explain the specificity and efficacy that Sindbis vectors demonstrate for tumor cells in vivo. Sindbis does not have to undergo genetic manipulation to target cancer cells or to be injected directly into tumors. Sindbis injected anywhere into a subject travels through the bloodstream to the target area (Tseng et al., Cancer Res. 64(18):6684-92 (2004). Sindbis can also be genetically engineered to carry one or more genes that suppress the immune response to the virus and/or genes that stimulate an immune response against the tumor such as, for example, antitumor cytokine genes such as interleukin-12 and interleukin-15 genes.

The virus may be chemically or biochemically pretreated (e.g., by treatment with a protease, such as chymotrypsin or trypsin) prior to administration to the neoplastic cells. Pretreatment with a protease removes the outer coat or capsid of the virus and may increase the infectivity of the virus. The virus may be coated in a liposome or micelle (Chandran and Nibert, J. of Virology 72(1):467-75 (1998)) to reduce or prevent an immune response from a mammal which has developed immunity to the virus. For example, the virion may be treated with chymotrypsin in the presence of micelle forming concentrations of alkyl sulfate detergents to generate a new infectious subvirion particle. The oncolytic virus may also be a reassortant virus or an ISVP.

The oncolytic virus may be a recombinant oncolytic virus. For example, the recombinant oncolytic virus results from the reassortment of genomic segments from two or more genetically distinct oncolytic viruses, also referred to herein as a reassortant. Reassortment of oncolytic virus genomic segments may occur following infection of a host organism with at least two genetically distinct oncolytic viruses. Recombinant viruses can also be generated in cell culture, for example, by co-infection of permissive host cells with genetically distinct oncolytic viruses. Optionally, the methods include the use of recombinant oncolytic virus resulting from reassortment of genome segments from two or more genetically distinct oncolytic viruses wherein at least one parental virus is genetically engineered, comprises one or more chemically synthesized genomic segment, has been treated with chemical or physical mutagens, or is itself the result of a recombination event. Optionally, the methods include the use of the recombinant oncolytic virus that has undergone recombination in the presence of chemical mutagens, including but not limited to dimethyl sulfate and ethidium bromide, or physical mutagens, including but not limited to ultraviolet light and other forms of radiation.

Optionally, the methods include the use of oncolytic viruses with mutations including (insertions, substitutions, deletions or duplications) in one or more genome segments. Such mutations can comprise additional genetic information as a result of recombination with a host cell genome, or that comprise synthetic genes such as, for example, genes encoding agents that suppress anti-viral immune responses.

Optionally, the oncolytic virus is a mutant oncolytic virus. For example, the oncolytic virus may be modified by incorporation of mutated coat proteins, such as for example, into the virion outer capsid. The mutant oncolytic virus is, optionally, a mutant reovirus. Mutant reoviruses as described herein can contain a mutation that reduces or essentially eliminates expression of a sigma3 polypeptide or that results in the absence of a functional sigma3 polypeptide as described in U.S. Publication No. 2008/0292594, which is incorporated by reference herein in its entirety. Optionally, the mutant reoviruses used in the provided methods are mutated as described in U.S. Pat. No. 7,803,385, which is incorporated by reference herein in its entirety.

A mutation as referred to herein can be a substitution, insertion or deletion of one or more nucleotides. Point mutations include, for example, single nucleotide transitions (purine to purine or pyrimidine to pyrimidine) or transversions (purine to pyrimidine or vice versa) and single- or multiple-nucleotide deletions or insertions. A mutation in a nucleic acid can result in one or more conservative or non-conservative amino acid substitutions in the encoded polypeptide, which may result in conformational changes or loss or partial loss of function, a shift in the reading frame of translation (frame-shift) resulting in an entirely different polypeptide encoded from that point on, a premature stop codon resulting in a truncated polypeptide (truncation), or a mutation in a virus nucleic acid may not change the encoded polypeptide at all (silent or nonsense). See, for example, Johnson and Overington, 1993, J. Mol. Biol. 233:716-38; Henikoff and Henikoff, 1992, Proc. Natl. Acad. Sci. USA 89:10915-19; and U.S. Pat. No. 4,554,101, for disclosure on conservative and non-conservative amino acid substitutions.

Mutations can be generated in the nucleic acid of an oncolytic virus using any number of methods known in the art. For example, site directed mutagenesis can be used to modify a reovirus nucleic acid sequence. One of the most common methods of site-directed mutagenesis is oligonucleotide-directed mutagenesis. In oligonucleotide-directed mutagenesis, an oligonucleotide encoding the desired change(s) in sequence is annealed to one strand of the DNA of interest and serves as a primer for initiation of DNA synthesis. In this manner, the oligonucleotide containing the sequence change is incorporated into the newly synthesized strand. See, for example, Kunkel, 1985, Proc. Natl. Acad. Sci. USA 82:488; Kunkel et al., 1987, Meth. Enzymol. 154:367; Lewis and Thompson, 1990, Nucl. Acids Res. 18:3439; Bohnsack, 1996, Meth. Mol. Biol. 57:1; Deng and Nickoloff, 1992, Anal. Biochem. 200:81; and Shimada, 1996, Meth. Mol. Biol. 57:157. Other methods are used routinely in the art to modify the sequence of a protein or polypeptide. For example, nucleic acids containing a mutation can be generated using PCR or chemical synthesis, or polypeptides having the desired change in amino acid sequence can be chemically synthesized. See, for example, Bang and Kent, 2005, Proc. Natl. Acad. Sci. USA 102:5014-9 and references therein.

Viruses can be purified using standard methodology. See, for example, Schiff et al., “Orthoreoviruses and Their Replication,” Ch 52, in Fields Virology, Knipe and Howley, eds., 2006, Lippincott Williams and Wilkins; Smith et al., 1969, Virology 39(4):791-810; and U.S. Pat. Nos. 7,186,542; 7,049,127; 6,808,916; and 6,528,305, which are incorporated by reference herein in their entireties. As used herein, purified viruses refer to viruses that have been separated from cellular components that naturally accompany them. Typically, viruses are considered purified when they are at least 70% (e.g., at least 75%, 80%, 85%, 90%, 95%, or 99%) by dry weight, free from the proteins and other cellular components with which they are naturally associated.

The provided methods include administering one or more additional agents to the subject. Optionally, the additional agent is a chemotherapeutic agent. Optionally, the additional agent is a cancer immunotherapeutic agent. Chemotherapeutic agents include, but are not limited to, alkylating agents, anthracyclines, taxanes, epothilones, histone deacetylase inhibitors, inhibitors of Topoisomerase I, inhibitors of Topoisomerase II, kinase inhibitors, monoclonal antibodies, nucleotide analogs and precursor analogs, peptide antibiotics, platinum-based compounds, retinoids, and vinca alkaloids and derivatives. Agents used in cancer immunotherapy are agents that stimulate the immune system to help a subject's immune system fight cancer. Cancer immunotherapeutic agents include cells like Dendritic cells and CAR-T cells, cytokines and antibodies. Optionally, the cancer immunotherapeutic agent is an immune checkpoint inhibitor. Optionally, the immune checkpoint inhibitor is an antibody. As discussed throughout, administration of an additional agent, e.g., a chemotherapeutic agent or cancer immunotherapeutic agent, can include administration of more than one additional agent to the subject, i.e., administration of a combination of additional agents.

As used herein, immune checkpoint inhibitor refers to any compound inhibits an immune inhibitory checkpoint protein. Inhibition can be reduction of function or complete blocking of function of the protein. Optionally, immune checkpoint inhibitors can be antibodies specifically recognizing immune checkpoint proteins. Immune checkpoint inhibitors are known and include peptides, antibodies, nucleic acid molecules and small molecules. Immune checkpoint refers to a molecule that is expressed by T cells that either enhances a signal (stimulatory checkpoint molecules) or decreases a signal (inhibitory checkpoint molecules). Immune checkpoint molecules are known to constitute immune checkpoint pathways similar to the CTLA-4 and PD-1 dependent pathways (see e.g. Pardoll, 2012. Nature Rev Cancer 12:252-264; Mellman et al, 2011. Nature 480:480-489). Examples of inhibitory checkpoint molecules include A2AR, B7-H3, B7-H4, BTLA, CTLA-4, CD277, IDO, KIR, PD-1, LAG-3, TIM-3 and VISTA.

Examples of anti-CTLA-4 antibodies are described in U.S. Pat. Nos. 5,811,097; 5,811,097; 5,855,887; 6,051,227; 6,207,157; 6,682,736; 6,984,720; and 7,605,238. For example, CTLA-4 antibodies include tremelimumab, (ticilimumab, CP-675,206) and ipilimumab (also known as 10D1, MDX-D010).

Examples of PD-1 and PD-L1 antibodies are described in U.S. Pat. Nos. 7,488,802; 7,943,743; 8,008,449; 8,168,757; 8,217,149, and PCT Published Patent Application Nos: WO03042402, WO2008156712, WO2010089411, WO2010036959, WO2011066342, WO2011159877, WO2011082400, and WO2011161699. PD-1 inhibitors include anti-PD-L1 antibodies and anti-PD-1 antibodies. Examples of PD-1 and PD-L1 inhibitors include nivolumab (MIX 1106, BMS 936558, ONO 4538); lambrolizumab (MK-3475 or SCH 900475); Pembrolizumab; Atezolizumab; Atezolizumab; Durvalumab; and Cemiplimab.

Other immune checkpoint inhibitors include lymphocyte activation gene-3 (LAG-3) inhibitors, such as IMP321, a soluble Ig fusion protein (Brignone et al, 2007, J. Immunol. 179:4202-4211). Immune checkpoint inhibitors include B7 inhibitors like B7-H3 and B7-H4 inhibitors. B7 inhibitors include anti-B7-H3 antibody MGA271 (Loo et al, 2012, Clin. Cancer Res. July 15 (18) 3834).

Optionally, the immune checkpoint inhibitor is an IDO inhibitor. Examples of IDO inhibitors are described in WO 2014150677. Examples of IDO inhibitors include 1-methyl-tryptophan (IMT), β-(3-benzofuranyl)-alanine, β-(3-benzo(b)thienyl)-alanine), 6-nitro-tryptophan, 6-fluoro-tryptophan, 4-methyl-tryptophan, 5-methyl tryptophan, 6-methyl-tryptophan, 5-methoxy-tryptophan, 5-hydroxy-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole a brassinin derivative, a thiohydantoin derivative, a β-carboline derivative or a brassilexin derivative. Optionally, the IDO inhibitor is selected from 1-methyl-tryptophan, β-(3-benzofuranyl)-alanine, 6-nitro-L-tryptophan, 3-Amino-naphtoic acid and β[3-benzo(b)thienyl]-alanine or a derivative or prodrug thereof. Examples of alkylating agents include, but are not limited to, nitrogen mustards, nitrosoureas, tetrazines, aziridines, cisplatins and derivatives, and non-classical alkylating agents. Specific examples of alkylating agents include, but are not limited to, mechlorethamine, cyclophosphamide, melphalan, chlorambucil, ifosfamide, busulfan, N-Nitroso-N-methylurea (MNU), carmustine (BCNU), lomustine (CCNU), semustine (MeCCNU), fotemustine, streptozotocin, dacarbazine, mitozolomide, temozolomide, thiotepa, mytomycin, diaziquone, cisplatin, carboplatin, oxaliplatin, procarbazine and hexamethylmelamine.

Other exemplary chemotherapeutic agents include but are not limited to 5-fluorouracil, mitomycin C, methotrexate, hydroxyurea, mitoxantrone, anthracyclins (e.g., epirubicin and doxurubicin), antibodies to receptors (e.g., herceptin, etoposide, pregnasome), hormone therapies (e.g., tamoxifen and anti-estrogens), interferons, aromatase inhibitors, progestational agents, and LHRH analogs. Suitable aromatase inhibitors include, but are not limited to, letrozole, anastrozole, exemestane, vorozole, formestane, fadrozole, testolactone, aminoglutethimide, 1,4,6-Androstatrien-3,17-dione, and 4-Androstene-3,6,17-trione.

Provided herein are pharmaceutical compositions comprising one or more oncolytic viruses. Also provided are pharmaceutical compositions comprising one or more chemotherapeutic agents. Optionally, the pharmaceutical compositions include one or more oncolytic viruses and one or more chemotherapeutic agents. Thus, the provided compositions can include a single agent or more than one agent.

The herein provided compositions are administered in vitro or in vivo in a pharmaceutically acceptable carrier. A pharmaceutically acceptable carrier can be a solid, semi-solid, or liquid material that can act as a vehicle, carrier or medium for the reovirus. Thus, compositions containing an oncolytic virus and/or one or more of the provided agents can be in the form of tablets, pills, powders, lozenges, sachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

Optionally, the compositions containing an oncolytic virus are suitable for infusion. For intravenous infusions, there are two types of fluids that are commonly used, crystalloids and colloids. Crystalloids are aqueous solutions of mineral salts or other water-soluble molecules. Colloids contain larger insoluble molecules, such as gelatin; blood itself is a colloid. The most commonly used crystalloid fluid is normal saline, a solution of sodium chloride at 0.9% concentration, which is close to the concentration in the blood (isotonic). Ringer's lactate or Ringer's acetate is another isotonic solution often used for large-volume fluid replacement. A solution of 5% dextrose in water, sometimes called D5W, is often used instead if the patient is at risk for having low blood sugar or high sodium.

Some examples of suitable carriers include phosphate-buffered saline or another physiologically acceptable buffer, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. A pharmaceutical composition additionally can include, without limitation, lubricating agents such as talc, magnesium stearate, and mineral oil; wetting agents; emulsifying and suspending agents; preserving agents such as methyl- and propylhydroxy-benzoates; sweetening agents; and flavoring agents. Pharmaceutical compositions can be formulated to provide quick, sustained or delayed release of a mutant reovirus after administration by employing procedures known in the art. In addition to the representative formulations described below, other suitable formulations for use in a pharmaceutical composition can be found in Remington: The Science and Practice of Pharmacy 22d edition Loyd V. Allen et al, editors, Pharmaceutical Press (2012). For preparing solid compositions such as tablets, a mutant reovirus can be mixed with a pharmaceutical carrier to form a solid composition. Optionally, tablets or pills can be coated or otherwise compounded to provide a dosage form affording the advantage of prolonged action. For example, a tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Liquid formulations that include a reovirus and/or agent for oral administration or for injection generally include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. These liquid or solid compositions may contain suitable pharmaceutically acceptable excipients as described herein. Such compositions can be administered by the oral or nasal respiratory route for local or systemic effect. Compositions in pharmaceutically acceptable solvents may be nebulized by use of inert gases. Nebulized solutions may be inhaled directly from the nebulizing device or the nebulizing device may be attached to a face mask tent or intermittent positive pressure breathing machine. Solution, suspension, or powder compositions may be administered, orally or nasally, from devices which deliver the formulation in an appropriate manner.

Another formulation that is optionally employed in the methods of the present disclosure includes transdermal delivery devices (e.g., patches). Such transdermal patches may be used to provide continuous or discontinuous infusion of the viruses and agents as described herein. The construction and use of transdermal patches for the delivery of pharmaceutical agents is well known in the art. See, for example, U.S. Pat. No. 5,023,252. Such patches can be constructed for continuous, pulsatile, or on-demand delivery of mutant reoviruses.

As described above, viruses and/or other agents can, if necessary, be coated in a liposome or micelle to reduce or prevent an immune response in a mammal that has developed immunity toward a virus or agent. Such compositions are referred to as immunoprotected viruses or agents. See, for example, U.S. Pat. Nos. 6,565,831 and 7,014,847.

In the provided methods, the oncolytic virus is administered, for example, systemically, in a manner so that it can ultimately contact the target tumor or tumor cells. The route by which the virus is administered, as well as the formulation, carrier or vehicle, depends on the location as well as the type of the target cells. A wide variety of administration routes can be employed. For example, for a solid tumor that is accessible, the virus can be administered by injection directly to the tumor. For a hematopoietic tumor, for example, the virus can be administered intravenously or intravascularly. For tumors that are not easily accessible within the body, such as metastases, the virus is administered in a manner such that it can be transported systemically through the body of the mammal and thereby reach the tumor (e.g., intravenously or intramuscularly). Alternatively, the virus can be administered directly to a single solid tumor, where it then is carried systemically through the body to metastases. The virus can also be administered subcutaneously, intraperitoneally, intrathecally or intraventricularly (e.g., for brain tumors), topically (e.g., for melanomas), orally (e.g., for oral or esophageal cancers), rectally (e.g., for colorectal cancers), vaginally (e.g., for cervical or vaginal cancers), nasally, by inhalation spray or by aerosol formulation (e.g., for lung cancers).

Optionally, the virus is administered continuously to a subject at least once per day or up to intermittently or continuously throughout the day on consecutive days, for a period of time for a first or subsequent round of treatment. Thus, the virus is administered, for example, to subjects by means of intravenous administration in any pharmacologically acceptable solution, or as an infusion over a period of time. For example, the substance may be administered systemically by injection (e.g., IM or subcutaneously) or taken orally daily at least once per day, or administered by infusion in a manner that results in the daily delivery into the tissue or blood stream of the subject. When the virus is administered by infusion over a period of time, the period of time is, for example, 1, 2, 3, 4, 5, 6, 7, 8, 9, 10, 12, or 24 hours, or any time between 1 and 24 hours, inclusive, or more. Optionally, the period of time is 5, 15, 30, 60, 90, 120, 150 or 180 minutes, or any time between 5 and 180 minutes, inclusive, or more. Thus, for example, the virus is administered by infusion for 60 minutes. Administrations can be repeated daily for 2, 3, 4, 5, 6, 7, 8, 9, 10, 14, 21, 28 days or any number of days between 2 and 28 days, inclusive, or longer.

The viruses as disclosed herein are administered in an amount that is sufficient (i.e., an effective amount) to effect the treatment of the cancer or proliferative disorder. A cancer or proliferative disorder is treated when administration of a treatment regimen including a virus to proliferating cells affects lysis (e.g., oncolysis) of the affected cells, resulting in a reduction in the number of abnormally, proliferating cells, a reduction in the size of a neoplasm, and/or a reduction in or elimination of symptoms (e.g., pain) associated with the proliferating disorder. As used herein, the term oncolysis means at least 10% of the proliferating cells are lysed (e.g., at least about 20%, 30%, 40%, 50%, or 75% of the cells are lysed). The percentage of lysis can be determined, for example, by measuring the reduction in the size of a neoplasm or in the number of proliferating cells in a mammal, or by measuring the amount of lysis of cells in vitro (e.g., from a biopsy of the proliferating cells). An effective amount of a virus used in a treatment regimen will be determined on an individual basis and may be based, at least in part, on the particular virus used; the individual's size, age, gender; and the size and other characteristics of the abnormally, proliferating cells. For example, for treatment of a human, approximately 10³ to 10¹² plaque forming units (PFU) of a virus are used, depending on the type, size and number of proliferating cells or neoplasms present. The effective amount can be, for example, from about 1.0 PFU/kg body weight to about 10¹⁵ PFU/kg body weight (e.g., from about 10² PFU/kg body weight to about 10¹³ PFU/kg body weight). Optionally, the effective amount is about 1×10⁸ to about 1×10¹² PFU or TCID50. Optionally, the effective amount is about 3×10¹⁰ to about 1×10¹⁰ TCID50.

Optimal dosages of viruses and therapeutic agents, and compositions and kits comprising viruses and agents depend on a variety of factors. The exact amount required will vary from subject to subject, depending on the species, age, weight and general condition of the subject, the severity of the disease being treated, the particular virus and its mode of administration. Thus, it is not possible to specify an exact amount for every composition or kit. However, an appropriate amount can be determined by one of ordinary skill in the art using only routine experimentation given the guidance provided herein.

Effective dosages and schedules for administering the treatment regimens may be determined empirically. For example, animal models for a variety of proliferative disorders can be obtained from the Jackson Laboratory, 600 Main Street, Bar Harbor, Me. 04609 USA. Both direct (e.g., histology of tumors) and functional measurements (e.g., survival of a subject or size of a tumor) can be used to monitor response to therapies. These methods involve the sacrifice of representative animals to evaluate the population, increasing the animal numbers necessary for the experiments. Measurement of luciferase activity in the tumor provides an alternative method to evaluate tumor volume without animal sacrifice and allowing longitudinal population-based analysis of therapy. The dosage ranges for the administration of compositions are those large enough to produce the desired effect in which the symptoms of the disease are affected. The dosage should not be so large as to cause adverse side effects, such as unwanted cross-reactions and anaphylactic reactions. The dosage can be adjusted by the individual physician in the event of any counterindications.

Dosages vary and are administered in one or more dose administrations, for example, daily, for one or several days. The provided viruses and therapeutic agents are administered in a single dose or in multiple doses (e.g., two, three, four, six, or more doses). For example, where the administration is by infusion, the infusion can be a single sustained dose or can be delivered by multiple infusions. Treatment may last from several days to several months or until diminution of the disease is achieved.

The provided methods may be further combined with other tumor therapies such as radiotherapy, surgery, hormone therapy and/or other immunotherapies. Suitable additional therapeutic agents include, but are not limited to, analgesics, anesthetics, analeptics, corticosteroids, anticholinergic agents, anticholinesterases, anticonvulsants, antineoplastic agents, allosteric inhibitors, anabolic steroids, antirheumatic agents, psychotherapeutic agents, neural blocking agents, anti-inflammatory agents, antihelmintics, antibiotics, anticoagulants, antifungals, antihistamines, antimuscarinic agents, antimycobacterial agents, antiprotozoal agents, antiviral agents, dopaminergics, hematological agents, immunological agents, muscarinics, protease inhibitors, vitamins, growth factors, and hormones. The choice of agent and dosage can be determined readily by one of skill in the art based on the given disease being treated.

Combinations of the provided viruses and therapeutic agents are administered either concomitantly (e.g., as an admixture), separately but simultaneously (e.g., via separate intravenous lines into the same subject), or sequentially (e.g., one of the compounds or agents is given first followed by the second). Thus, the term combination is used to refer to concomitant, simultaneous, or sequential administration of two or more agents.

When one compound is administered prior to another compound, the first compound is administered minutes, hours, days, or weeks prior to administration of the second compound. For example, the first compound can be administered at 1, 2, 3, 4, 5, 6, 7, 8, 9, 10, 12, 24, 36, 48, 60, or 72 hours, or any time between 1 and 72 hours, inclusive, prior to administration of a second compound. Optionally, the first compound is administered more than 72 hours prior to the second compound. By way of another example, the first compound can be administered at 1, 5, 15, 30, 60, 90, 120, 150 or 180 minutes, or any time between 1 and 180 minutes, inclusive, prior to administration of a second compound. Optionally, the first compound is administered at 1, 2, 3, 4, 5, 6, 7, 14, 21, or 28 days, or any amount in between 1 and 28, inclusive, days prior to administration of the second compound. Optionally, the first compound is administered more than 28 days prior to the second compound.

Oncolytic viruses or a pharmaceutical composition comprising such viruses can be packaged into a kit. The kit also includes one or more additional agents or pharmaceutical compositions comprising the additional agents. The kit can include chemotherapeutic agents or cancer immunotherapeutic agents. Optionally, the kit includes an immune checkpoint inhibitor. The oncolytic viruses and/or additional agents and pharmaceutical compositions containing the same can be packaged in one or more containers. When the kits contain pharmaceutical compositions, the pharmaceutical compositions can be formulated in a unit dosage form. The term unit dosage forms refers to physically discrete units suitable as unitary dosages for human subjects and other mammals, each unit containing a predetermined quantity of an oncolytic virus or other agent, e.g., immune checkpoint inhibitor calculated to produce the desired therapeutic effect in association with a suitable pharmaceutically acceptable carrier. Optionally, the kit includes a reovirus and an immune checkpoint inhibitor.

The oncolytic virus in the provided kits can be any of the oncolytic viruses described herein. The provided kits can include more than one dose of the oncolytic virus. Optionally, each dose of oncolytic virus comprises approximately 10³ to 10¹² plaque forming units (PFU) of the oncolytic virus. Optionally, each dose comprises approximately 10⁸ to 10¹² PFU of the oncolytic virus. Optionally, each dose comprises approximately 10⁸ to 10¹² TCID50 of the oncolytic virus. Optionally, each dose comprises approximately 1×10¹⁰ to 3×10¹⁰ TCID50 of the oncolytic virus.

As used herein the terms treatment, treat, treating or ameliorating refers to a method of reducing the effects of a disease or condition or symptom of the disease or condition. Thus in the disclosed method, treatment can refer to a 10%, 20%, 30%, 40%, 50%, 60%, 70%, 80%, 90% or 100% reduction or amelioration in the severity of an established disease or condition or symptom of the disease or condition. For example, the method for treating cancer is considered to be a treatment if there is a 10% reduction in one or more symptoms of the disease in a subject as compared to control. Thus the reduction can be a 10, 20, 30, 40, 50, 60, 70, 80, 90, 100% or any percent reduction in between 10 and 100 as compared to native or control levels. It is understood that treatment does not necessarily refer to a cure or complete ablation of the disease, condition or symptoms of the disease or condition.

As used herein, the term subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The term does not denote a particular age or sex. Thus, adult and newborn subjects, whether male or female, are intended to be covered. As used herein, patient or subject may be used interchangeably and can refer to a subject with a disease or disorder. The term patient or subject includes human and veterinary subjects.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if an inhibitor is disclosed and discussed and a number of modifications that can be made to a number of molecules including the inhibitor are discussed, each and every combination and permutation of the inhibitor, and the modifications that are possible are specifically contemplated unless specifically indicated to the contrary. Likewise, any subset or combination of these is also specifically contemplated and disclosed. This concept applies to all aspects of this disclosure including, but not limited to, steps in methods of using the disclosed compositions. Thus, if there are a variety of additional steps that can be performed it is understood that each of these additional steps can be performed with any specific method steps or combination of method steps of the disclosed methods, and that each such combination or subset of combinations is specifically contemplated and should be considered disclosed.

Throughout this application, various publications are referenced. The disclosures of these publications in their entireties are hereby incorporated by reference into this application.

A number of aspects have been described. Nevertheless, it will be understood that various modifications may be made. Furthermore, when one characteristic or step is described it can be combined with any other characteristic or step herein even if the combination is not explicitly stated. Accordingly, other aspects are within the scope of the claims.

### EXAMPLES

### Example 1. Analysis of T Cell Repertoire Upon Treatment with Pelareorep and Chemotherapy in Patients with Pancreatic Adenocarcinoma

Reovirus Serotype 3-Dearing Strain (pelareorep) is a non-enveloped human reovirus that has been shown to induce tumor lysis and innate and adaptive immune responses, which lead to an inflamed phenotype and antitumor activity. A study was performed with pelareorep and chemotherapy in combination with pembrolizumab in patients with advanced (unresectable or metastatic) histologically confirmed pancreatic adenocarcinoma that progressed after (or did not tolerate) first-line therapy. The study characterized pelareorep given intravenously in combination with pembrolizumab and one of the three chemotherapy backbone regimens, Gemcitabine, Irinotecan or Leucovorin/5-fluorouracil (5-FU), in treatment cycles of which one was repeated every 3 weeks until disease progression.

Experimental Design: T cells were analyzed by the immunoSEQ assay (Adaptive Biotechnologies®; Seattle, Wash.) at C1D1 and C2D1 (approximately 3 weeks later) in a set of nine subjects. More specifically, genomic DNA was obtained from samples of peripheral blood mononuclear cells (PBMC) at each time point. The following table is a summary of the analysis. Values shown are the average with the range (minimum-maximum) in parentheses.

| | C1D1 | | C2D1 | |
|---|---|---|---|---|
| n | 9 | | 9 | |
| Total Cells | 131524 | (50745-256220) | 182965 | (67886-293568) |
| Total T Cells | 46221 | (2326-111288) | 68469 | (25496-116290) |
| T Cell Fraction | 0.322 | (0.042-0.548) | 0.386 | (0.147-0.678) |
| Unique T Cells | 25756 | (2267-77002) | 39421 | (13377-94861) |
| Productive | 0.155 | (0.015-0.311) | 0.147 | (0.049-0.296) |
| Clonality | | | | |
| Max Produc- | 0.061 | (0.009-0.155) | 1276 | (434-2001) |
| tive Frequency | | | | |
| Input DNA | 0.047 | (0.011-0.107) | 1621 | (472-2001) |
| (ng) | | | | |

Clonality and Diversity were calculated using the equations below:

$$Diversity = H = - {\sum }_{i = 1}^{N} {p}_{i} {log}_{2} ( {p}_{i} )$$

$$Clonality = 1 - \frac{H}{{log}_{2} ( N )} .$$

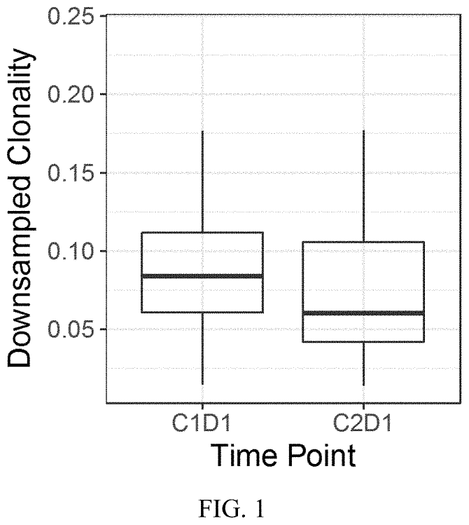

Clonality is robust to sampling depth within a range. Due to the two orders of magnitude difference, all samples were downsampled to the minimum templates count of 2581. As shown in FIG. 1, there was a trend towards decreased peripheral clonality over treatment using a paired Wilcox rank sum test. PWRS<0.01.

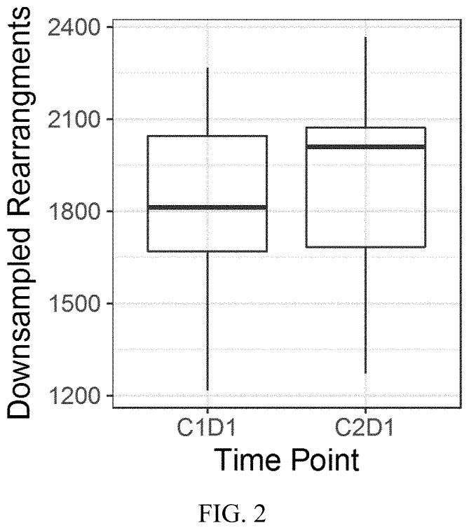

Clonality is often inverse to diversity. Diversity is the number of unique rearrangements given 2581 templates. As shown in FIG. 2, there was a trend towards increased peripheral diversity over treatment using a paired Wilcox rank sum test, although not as significant as clonality.

Log-rank (Mantel-Cos) analysis was performed with progression free survival and overall survival. Clonality was scaled to a unit of 0.1. Diversity was scaled to a unit of 100.

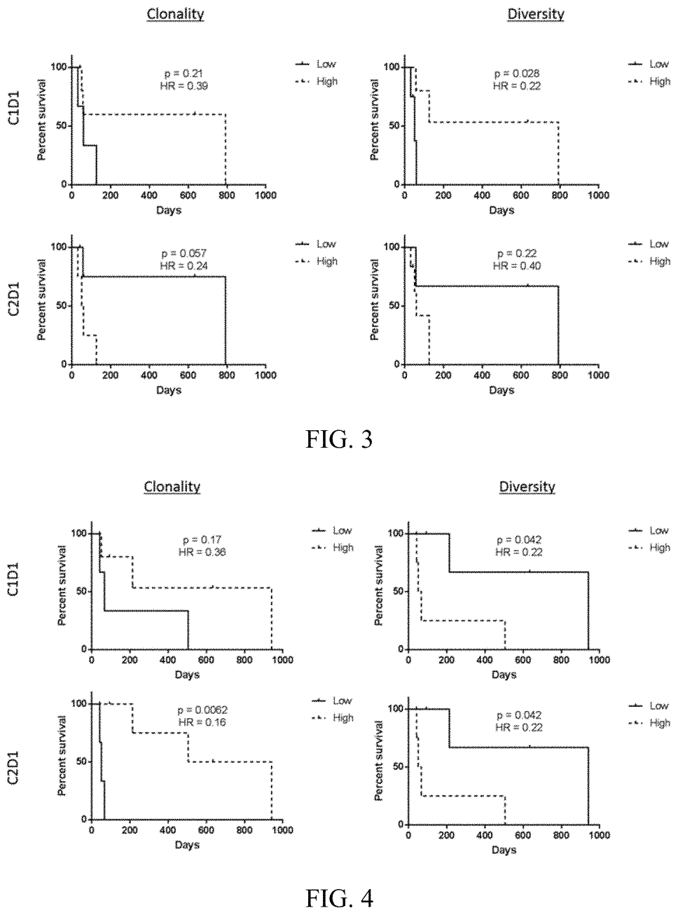

As shown in FIG. 3, clonality and diversity are correlated with progression free survival and show a stronger p-value at C1D1. Higher peripheral clonality and lower diversity are associated with longer progression free survival.

As shown in FIG. 4, clonality and diversity are correlated with overall survival and show a stronger p-value at C2D1. Higher peripheral clonality and lower diversity are associated with better outcome.

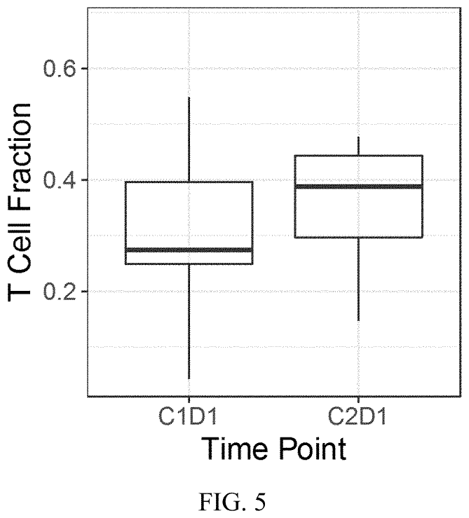

Peripheral T Cell Fraction is the number of T cells for total nucleated cells. As shown in FIG. 5, there was a trend toward a slight increase in the peripheral T cell fraction, however, there are patients moving in both directions and overall trend is not significant.

A binomial metric with false discovery rate (FDR) correction was used to look at differential clone frequency. This calculates metrics for expanded clones and repertoire overlap/similarity. Expanded clones were both new and existing.

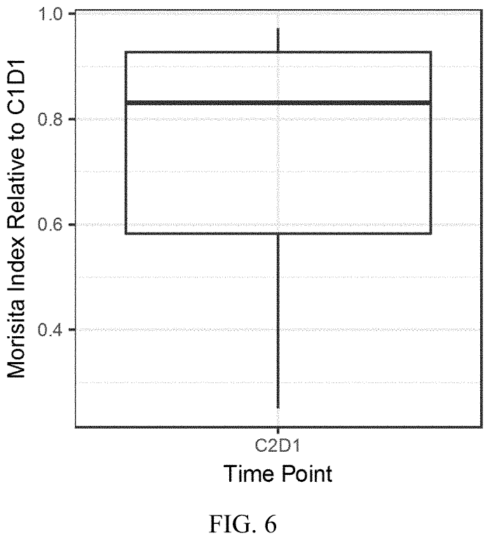

The Morisita Index takes into account both repertoire overlap and clonal frequencies between the two samples. A perfectly identical repertoire is 1, and two completely disparate samples would be 0. Normal variation over a month is about 0.9-0.95. As shown in FIG. 6, the median Morisita index between C2D1 and C1D1 is 0.83 with 3 samples below 0.6. This suggests significant peripheral repertoire turnover.

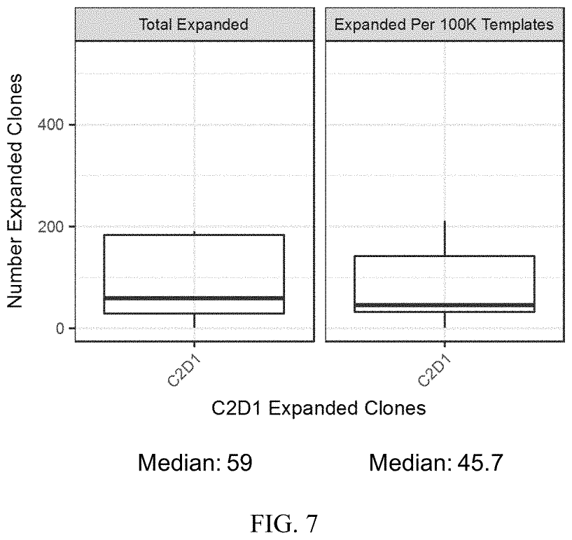

Peripherally expanded clones were determined between C1D1 and C2D1. Due to the high variation in template counts, expanded clones per 100,000 cumulative templates were reported. Normal variation over 4 weeks is about 5-10 expanded clones. As shown in FIG. 7, median values are greater than 40 in both cases. Only one sample had less than 18 expanded clones.

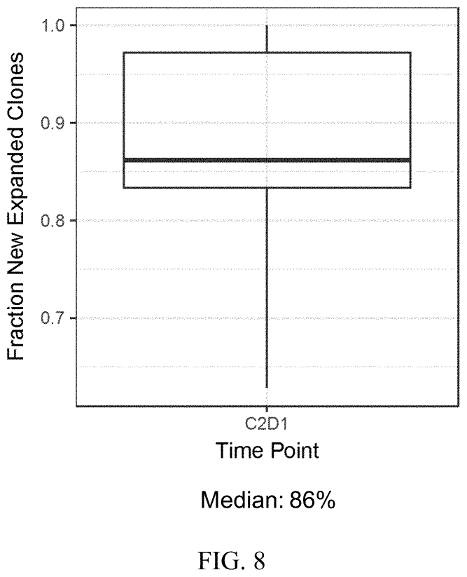

Peripherally expanded clones can be either expansion of existing clones or newly identified clones (i.e. undetected in the first time point). As shown in FIG. 8, most peripheral clonal expansion was observed from new clones.

In summary, peripheral clonality decreases and unique rearrangements increase between C1D1 and C2D1, consistent with a general increase in diversity. Higher peripheral clonality and lower diversity are associated with better overall survival. High levels of peripheral repertoire turnover occur between C1D1 and C2D1. Repertoire turnover is accompanied by significant clonal expansion, mostly by increase in “new” clones (clones that were undetected in C1D1).

### Example 2. Analysis of T Cell Repertoire Upon Treatment with Pelareorep and an Aromatase Inhibitor or Checkpoint Inhibitor in Patients with Breast Cancer

To study the T Cell Response and changes within the tumor microenvironment (TME), women with early breast cancer were divided into two groups (6 patients each) and administered pelareorep in combination with letrozole or atezolizumab. Patients were treated with pelareorep on days 1, 2, 8, and 9. Letrozole was administered daily starting on day 3 while atezolizumab was administered once on day 3. Tumor biopsies were collected at diagnosis, day 3, and day ˜21. The primary endpoint of the study was CelTIL score. CelTIL score is a metric for quantifying the changes in tumor cellularity and infiltration of TILs, where an increase in CelTIL is associated with a favorable response to treatment (Nuciforo, et al., Ann. Oncol. 29:170-77 (2018). Tumor tissue was examined for pelareorep replication, and changes to the TME were assessed by immunohistochemistry and TCR-immunosequencing (immune SEQ assay). Peripheral blood was also examined by TCR-immunosequencing.

Analysis of CelTIL show an increase in four of the six patients to date. Productive viral replication in day 3 and day 21 biopsies was very high, as measured by in situ detection of viral capsid protein in tumor cells. Immunohistochemistry analysis revealed an increase in CD8+ T cells and upregulation of PDL1 on day 3 and day 21 biopsies for all patients. Overall, the degree of viral replication was consistent with changes in CelTIL and changes within the TME. TCR-seq from blood showed that levels of T cell clonality correlated with changes in the TME and CelTIL. Thus, higher T cell peripheral clonality correlated with higher CelTIL indicating patients were more responsive to treatment.

1. A method of treating cancer in a subject, the method comprising:
(i) administering to the subject a one or more doses of an oncolytic virus;
(ii) selecting a subject with a T-cell population exhibiting high peripheral clonality after treatment with the one or more doses of the oncolytic virus; and
(iii) administering to the subject with a T-cell population exhibiting high peripheral clonality one or more subsequent doses of the oncolytic virus.

2. The method of claim 1, wherein the peripheral clonality is greater than 0.06.

3. The method of claim 1, wherein the subject also has a T cell population with low diversity.

4. The method of claim 3, wherein the T cell population diversity is less than 1800 rearrangements.

5. The method of claim 1, wherein the cancer is an adenocarcinoma.

6. The method of claim 1, wherein the cancer is breast cancer or pancreatic adenocarcinoma.

7. The method of claim 1, wherein approximately 10³ to 10¹² plaque forming units (PFU) of the oncolytic virus is administered to the subject.

8. The method of claim 1, wherein approximately 10⁸ to 10¹² PFU of the oncolytic virus is administered to the subject.

9. The method of claim 1, wherein approximately 10⁸ to 10¹² TCID50 of the oncolytic virus is administered to the subject.

10. The method of claim 1, wherein the oncolytic virus is administered as an intravenous infusion.

11. The method of claim 1, further comprising administering one or more additional therapeutic agents to the subject.

12. The method of claim 11, wherein the additional therapeutic agent is a chemotherapeutic agent.

13. The method of claim 11, wherein the additional agent is an immune checkpoint inhibitor or an aromatase inhibitor.

14. The method of claim 13, wherein the immune checkpoint inhibitor is a PD-1 or PD-L1 inhibitor.

15. The method of claim 13, wherein the immune checkpoint inhibitor is selected from the group consisting of nivolumab, lambrolizumab, pembrolizumab, atezolizumab, avelumab, durvalumab, and cemiplimab.

16. The method of claim 1, wherein selection of the subject with a T cell population with high peripheral clonality and low diversity results in longer progression free survival or overall survival of the subject treated with the oncolytic virus as compared subjects without selection or as compared to subjects lacking a T cell population with high peripheral clonality and low diversity after one or more doses of the oncolytic virus.

17. The method of claim 1, wherein the oncolytic virus is selected from the group consisting of a reovirus, a Newcastle disease virus (NDV), a vesicular stomatitis virus (VSV), an adenovirus, a vaccinia virus, a parapox orf virus, a Sindbis virus, and a herpes simplex virus.

18. The method of claim 17, wherein the reovirus is a mammalian reovirus.

19. The method of claim 18, wherein the reovirus is a human reovirus.

20. The method of claim 18, wherein the reovirus is selected from the group consisting of serotype 1 reoviruses, serotype 2 reoviruses, serotype 3 reoviruses.

21. The method of claim 20, wherein the reovirus is a serotype 3 reovirus.

22. The method of claim 21, wherein the serotype 3 reovirus is a Dearing strain reovirus.

23. The method of claim 18, wherein the reovirus is deposited as IDAC Accession No. 190907-01.

24. The method of claim 18, wherein the reovirus comprises a lambda-3 polypeptide having one or more amino acid modifications, a sigma-3 polypeptide having one or more amino acid modifications, a mu-1 polypeptide having one or more amino acid modifications, a mu-2 polypeptide having one or more amino acid modifications, or any combination thereof.

25. The method of claim 18, wherein the reovirus comprises one or more of the following polypeptides:
a sigma-3 polypeptide having one or more amino acid modifications, wherein the one or more amino acid modifications are selected from the group consisting of a Leu at residue 14, a Lys at residue 198, or any combination thereof, numbered relative to GenBank Accession No. K02739, wherein when the amino acid sequence comprises a Leu at residue 14, the amino acid sequence further comprises at least one additional modification in the amino acid sequence; a mu-1 polypeptide having at least one amino acid modification, wherein the at least one amino acid modification comprises an Asp at residue 73 numbered relative to GenBank Accession No. M20161.1;
a lambda-3 polypeptide having one or more amino acid modifications, wherein the one or more amino acid modifications are selected from the group consisting of a Val at residue 214, an Ala at residue 267, a Thr at residue 557, a Lys at residue 755, a Met at residue 756, a Pro at residue 926, a Pro at residue 963, a Leu at residue 979, an Arg at residue 1045, a Val at residue 1071, or any combination thereof, numbered relative to GenBank Accession No. M24734.1, wherein when the amino acid sequence comprises a Val at residue 214 or a Val at residue 1071, the amino acid sequence further comprises at least one additional modification in the amino acid sequence; or
a mu-2 polypeptide having at least one amino acid modification, wherein the at least one amino acid modification comprises a Ser at residue 528 numbered relative to GenBank Accession No. AF461684.1.

26. The method of claim 18, wherein the reovirus comprises one or more of the following genome segments:
a S4 genome segment having one or more nucleic acid modifications, wherein the one or more nucleic acid modifications in the S4 genome segment are selected from the group consisting of an A at position 74 and an A at position 624, numbered relative to GenBank Accession No. K02739;
a M2 genome segment having at least one nucleic acid modification, wherein the at least one nucleic acid modification comprises a C at position 248, numbered relative to GenBank Accession No. M20161.1;
a L1 genome segment comprising one or more nucleic acid modifications, wherein the one or more nucleic acid modifications are selected from the group consisting of a T at position 660, a G at position 817, an A at position 1687, a G at position 2283, an ATG at positions 2284-2286, a C at position 2794, a C at position 2905, a Cat position 2953, an A at position 3153, a G at position 3231, numbered relative to GenBank Accession No. M24734.1; or
a M1 genome segment having at least one nucleic acid modification, wherein the at least one nucleic acid modification comprises a T at position 1595, numbered relative to GenBank Accession No. AF461684.1.

27. The method of claim 18, wherein the reovirus is a recombinant reovirus.

28. The method of claim 18, wherein the reovirus is a modified reovirus.
